(19) United States
(12) Patent Application Publication
(10) Pub. No.: US 2026/0176383 A1
(43) Pub. Date: Jun. 25, 2026

(54) ANTIOZONANT COMPOSITIONS AND METHODS

(71) Applicant: The United States of America, as represented by the Secretary of Agriculture, Washington, DC (US)

(72) Inventors: William M Hart-Cooper, Richmond, CA (US); Elliot C. Rossomme, San Francisco, CA (US); Colleen M McMahan, Sausalito, CA (US); Anh Tran, HAYWARD, CA (US); William J. Orts, BURLINGAME, CA (US)

(21) Appl. No.: 19/426,609

(22) Filed: Dec. 19, 2025

Related U.S. Application Data
(60) Provisional application No. 63/737,900, filed on Dec. 23, 2024.

(51) Int. Cl. C08C 19/28; B60C 1/00; C07C 251/12; C07C 251/16; C07C 251/24; C07C 337/06; C07D 207/325; C07D 207/335; C07D 207/50; C07D 209/08; C07D 209/14; C07D 403/12; C07D 495/04

(52) U.S. Cl. CPC C08C 19/28; B60C 1/0025; C07C 251/12; C07C 251/16; C07C 251/24; C07C 337/06; C07D 207/325; C07D 207/335; C07D 207/50; C07D 209/08; C07D 209/14; C07D 403/12; C07D 495/04

(57) ABSTRACT

Compositions and methods of making and using the compositions are described exemplifying compounds formed from amine-functionalized indoles and other amines for uses such as rubber antiozonants.

### CROSS REFERENCE TO RELATED APPLICATIONS

This application claims the benefit of U.S. Provisional Application No. 63/737,900, titled “ANTIOZONANT COMPOSITIONS AND METHODS” filed Dec. 23, 2024, which is incorporated herein by reference in its entirety.

### FIELD OF THE INVENTION

The invention relates to antiozonant compositions and methods.

### BACKGROUND OF THE INVENTION

Rubber polymers contain backbone unsaturation essential to their low glass transition and flexible elastomeric physical properties. To create durable rubber products, these sites (C═C) are targeted by added vulcanization agents (sulfur, peroxide, etc.) that chemically crosslink the polymer chains under heat, creating a thermoset composite that maintains flexibility and integrity at high temperatures and loads. However, crosslinking is not 100% efficient, and residual backbone unsaturation creates vulnerability to degradation by light, oxygen, ozone, and radiation. Antidegradants are added to rubber compounds, such as those used in tires, to protect the residual unsaturation from degradation (chain scission), by providing chemical functionality that is more reactive than the rubber, and/or can scavenge radicals produced. In tire compounds, one critical class of antidegradants is the antiozonants, the most successful of which are the p-phenylenediamines (PPDs), especially 6PPD (N-(1,3-dimethylbutyl)-N′-phenyl-p-phenylenediamine).

The use of 6PPD in tires has come under scrutiny due to the aquatic toxicity of 6PPD quinone (6PPDQ), a transformation product that forms upon the reaction of 6PPD with ozone. The publication of Tian, et al. (2021) indicates that 6PPD and its transformation products are extremely toxic to aquatic life, and in particular is responsible for mass die-off of coho salmon in the Pacific Northwest. Both 6PPD and 6PPDQ have been detected in roadway runoff and exhibit varying degrees of toxicity to fish of economic, ecological, and cultural significance (Table 1). As a result, the replacement of 6PPD with safer antiozonants has become a pressing problem for environmental agencies, tire manufacturers, and chemical suppliers alike. The scope of this problem is significant, with 50 to 100 million pounds of 6PPD being sold in U.S. market annually.

Finding effective 6PPD replacements is a significant challenge as multiple chemical and physical requirements must be satisfied. First, alternatives must exhibit a high degree of reactivity toward ozone and other oxidative species, like alkyl and alkoxyl radicals, in order to prevent rubber degradation. Candidates must also possess desired diffusivity properties in order to effectively migrate through rubber layers in the tire to maintain working concentrations of anti-degradant at the surface of the tire. They must also be compatible with the chemical and physical conditions of tire processing, which includes withstanding elevated temperatures (up to 190° C.) used in tire manufacturing and stability in the presence of vulcanization and curing agents. The design of rubber antiozonants with reduced aquatic toxicity is challenging, because these chemicals function through their high reactivity. Additionally, development of safer rubber anti-degradants has become a critical problem as state and federal agencies begin to consider regulations for these chemicals.

There thus exists an ongoing need to develop efficacious, novel, and safe compositions and methods to alleviate these problems, which are also cost-effective and easily adaptable for widespread use.

### SUMMARY OF THE INVENTION

Described herein in various embodiments are compositions having antiozonant properties and methods of using and making them.

In one embodiment, a method of treating a rubber polymer to protect it against damage by ozone comprising, treating the polymer with an effective amount of a composition comprising a compound of formula I is described.

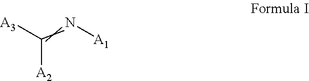

wherein A1 is

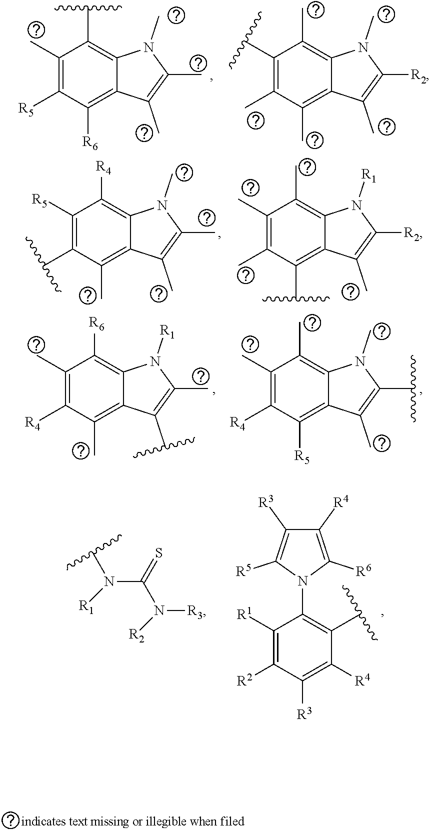

n-butyl, sec-butyl, iso-butyl, tert-butyl groups or a monocyclic heteroaryl group, each of which is optionally substituted by one or more of R1, R2, R3, R4, R5, R6,
wherein, A2 and A3 are independently selected from
a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO2, OH, NR7R8, acyl, alkyl ether or aryl ether substituents or an imine functional group bearing an A1, A2 or A3 substituent; wherein the aryl substituents are optionally substituted with one or more F, Cl, Br, I, NO2, OH, NR7R8, acyl, alkyl ether or aryl ether substituents;
wherein R1, R2, R3, R4, R5, R6 are independently selected from H, alkyl, unsaturated alkyl, F, Cl, Br, I, NO2, OH, amino, acyl, alkyl ether or aryl ether, monocyclic or polycyclic aryl or heteroaryl group;
and wherein R7, R8 are H or alkyl.

In one embodiment, a method of treating a rubber polymer to protect it against damage by ozone is described wherein the composition includes compounds of formula I which are cis or trans isomers of one or more CN imine bonds or a mixture of the isomers.

In one embodiment a rubber polymer formulation is described comprising an antiozonant compound of formula I.

In one embodiment of the rubber polymer formulation, the antiozonant compound is present in an amount from 0.1 to 10 parts per 100 parts by weight of rubber.

In one embodiment of a rubber polymer formulation described above, the formulation includes at least one antiozonant compound of Formula I described above in a vulcanized elastomeric article. In another embodiment, the vulcanized elastomeric article is a vehicle rubber tire.

In one embodiment, a composition comprising a compound of formula I is described.

### BRIEF DESCRIPTION OF THE DRAWINGS

FIG. 1 Toxicity of 6PPD and 6PPQ to various aquatic species

FIG. 2 General reaction scheme for Schiff base condensation.

FIG. 3 Condensation of 5-aminoindole and 2-methoxycinnamaldehyde

FIG. 4 Condensation of 6-aminoindol and citral

FIG. 5 Proton NMR spectrum for SB of 5-aminoindole and 2-methoxycinnamladehyde

FIG. 6 FTIR spectrum of Schiff base of 5-aminoindole and 2-methoxycinnamaldeyde

FIG. 7 Proton NMR spectrum of the Schiff base of 6-aminoindole and citral

FIG. 8 Percent efficiency of Schiff base antiozonants as determined through solution ozonation of Guayule natural rubber. Scale is normalized to performance of rubber without antidegradant (0%) and rubber treated with 6PPD (100%).

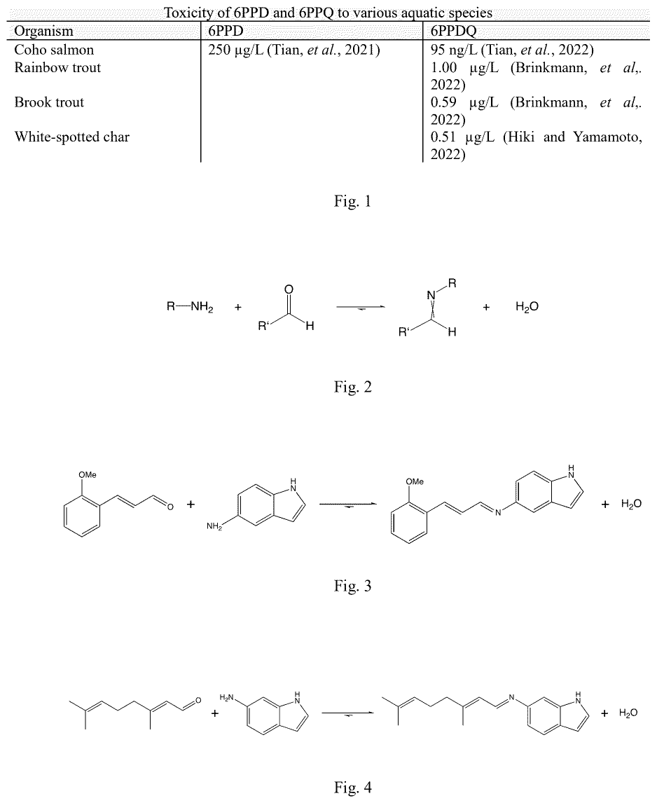

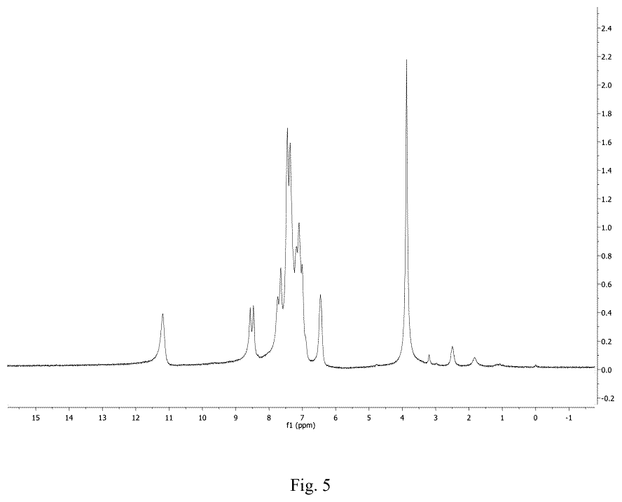

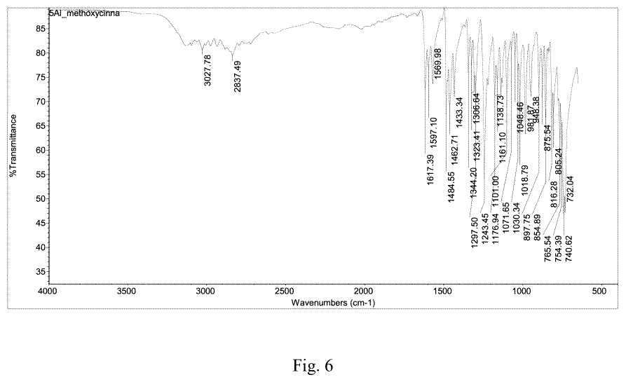

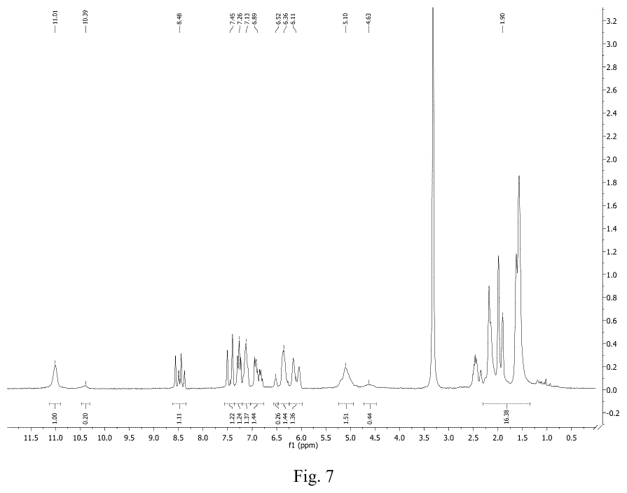

### DETAILED DESCRIPTION

Described herein in various embodiments are compositions and methods of using compositions wherein reversible imine products readily self-assemble from subcomponents and provide one or more of antiozonant, antioxidant, and dye properties, due to their vibrant colors. Imine bond incorporation in some embodiments provides dual functionality by (a) enabling aqueous dissociation to low toxicity byproducts and (b) providing a sacrificial functional group that reacts with ozone without forming a quinone. In other embodiments the imine functional group of formula I is reduced to a its amine derivative product molecule which is used as an antiozonant, antioxidant or dye.

In one embodiment a method of protecting rubber polymer from ozone degradation by treatment with an effective amount of a composition of the formula below is described:

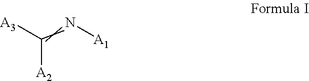

wherein A1 is

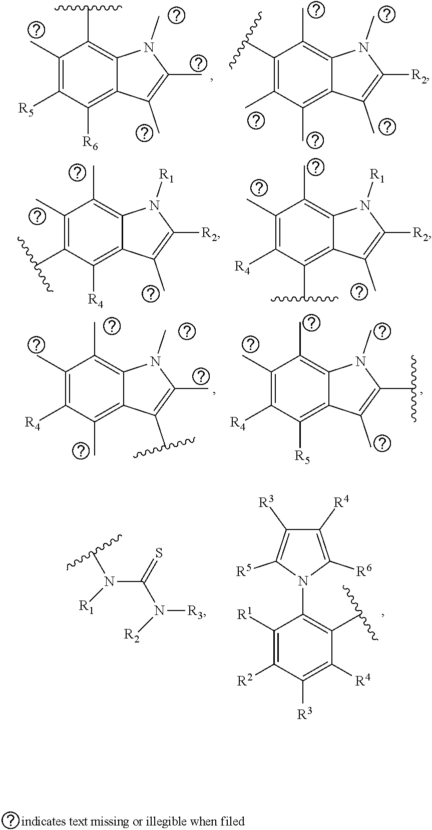

n-butyl, sec-butyl, iso-butyl, tert-butyl groups or a monocyclic heteroaryl group each of which is optionally substituted by one or more of R1, R2, R3, R4, R5, R6,
wherein, A2 and A3 are independently selected from
a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, NR₇R₈, acyl, alkyl ether or aryl ether substituents;
wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group;
and wherein R₇, R₈ are H or alkyl.

In one embodiment the composition comprises compounds with cis or trans isomers of a CN imine bond or a mixture of the isomers.

In various embodiments, compositions having effective antiozonant properties are formed by the condensation of amines exemplified by 4-aminoindole (4AI), 5-aminoindole (5AI), 6-aminoindole (6AI) and their derivatives with a substituted or unsubstituted aliphatic or aromatic aldehyde.

In various embodiments, compositions having effective antiozonant properties are formed by the condensation of compounds exemplified by 4-aminoindole, 5-aminoindole or 6-amino indole, other amine bearing molecules and their derivatives with aldehydes.

In one embodiment an antiozonant composition is described comprising of:

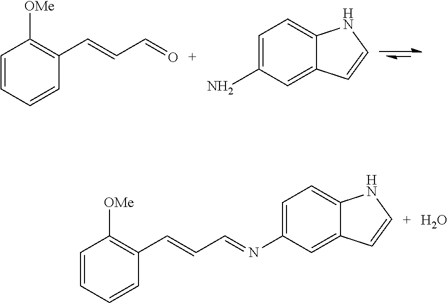

In one embodiment, the antiozonant composition above consists predominantly of the Schiff base product.

In one embodiment an antiozonant composition comprising at least one imine or Schiff base structure below is described:

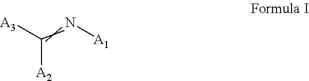

wherein A1 is
1.

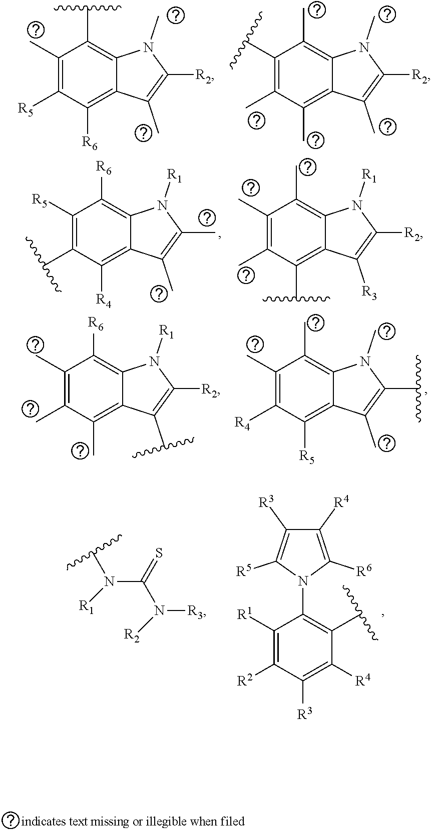

n-butyl, sec-butyl, iso-butyl, tert-butyl groups or a monocyclic heteroaryl group, optionally substituted by one or more of R₁, R₂, R₃, R₄, R₅, R₆,
wherein, A2 and A3 are independently selected from a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl.

In one embodiment, the antiozonant composition above consists predominantly of the imine product and includes its amine, aldehyde precursors.

In one embodiment a method of protecting rubber polymer from ozone degradation by treatment with an effective amount of a composition of the formula 1 below is described:

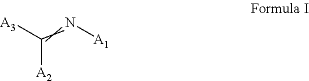

wherein A1 is

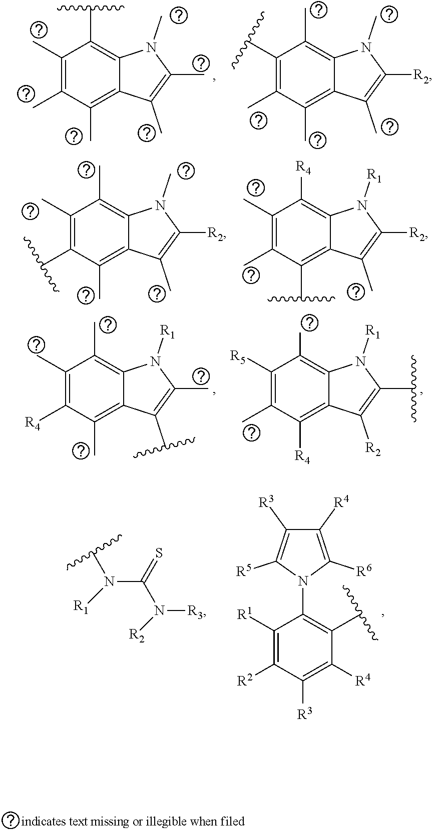

n-butyl, sec-butyl, iso-butyl, tert-butyl groups or a monocyclic heteroaryl group each of which is optionally substituted by one or more of R₁, R₂, R₃, R₄, R₅, R₆,
wherein, A2 and A3 are independently selected from
a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, NR₇R₈, acyl, alkyl ether or aryl ether substituents;
wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group;
and wherein R₇, R₈ are H or alkyl.

In one embodiment, the method comprises a composition of at least one compound of formula I with cis or trans isomers of a CN imine bond or a mixture of the isomers and may include its amine, aldehyde precursors. In various embodiments, methods of protecting rubber polymers from ozone degradation by treatment with compositions having antiozonant properties by the condensation of an amine exemplified by 5-aminoindole with exemplified aldehyde 2-methoxycinnamladehyde (6) to form N-(1H-indol-5-yl)-3-(2-methoxyphenyl) prop-2-en-1-imine.

In various embodiments, methods of protecting rubber polymers from ozone degradation by treatment with compositions having effective antiozonant properties are described. The antiozonant compositions are exemplified by the imine or Schiff Base formula I and are formed by the exemplified condensation of 5-aminoindole with terephthalaldehyde to form the imine product.

In preferred embodiments, antiozonant compounds described herein are exemplified by the compounds shown below. The CN double bond(s) in the structures shown are crossed to indicate that cis or trans isomers or mixtures of the geometric isomers of the compounds is represented in the figures:

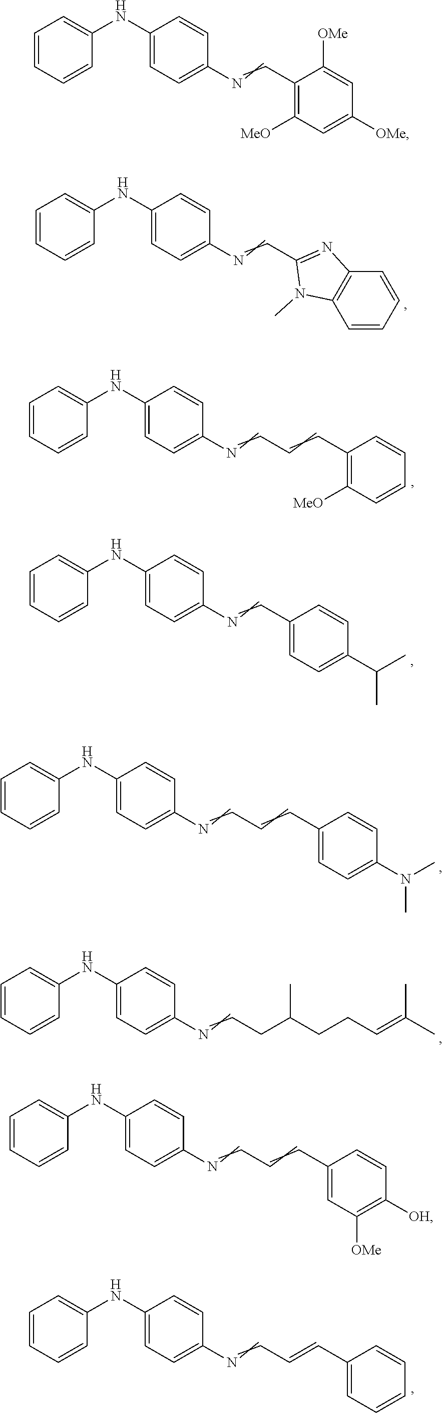

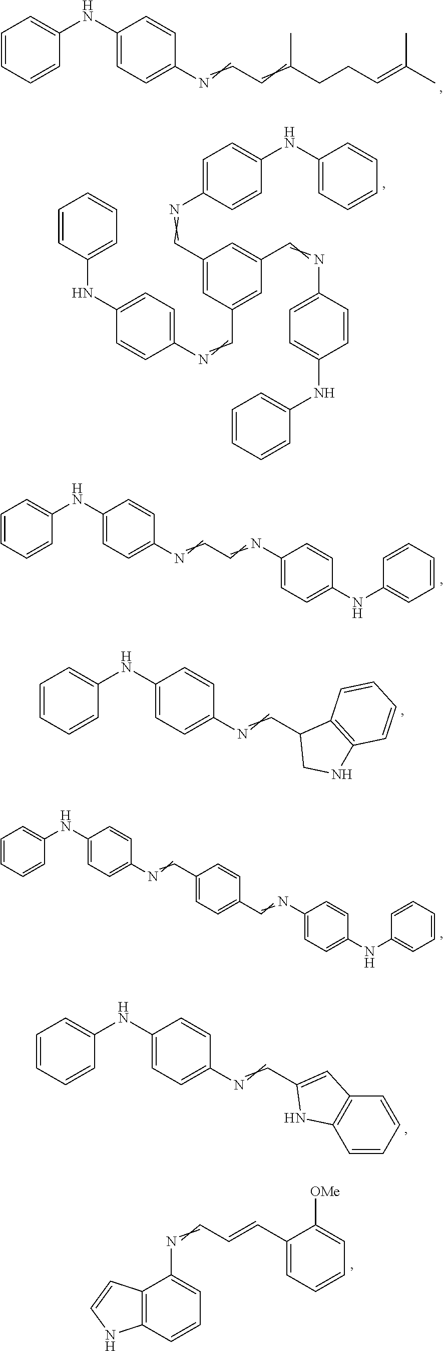

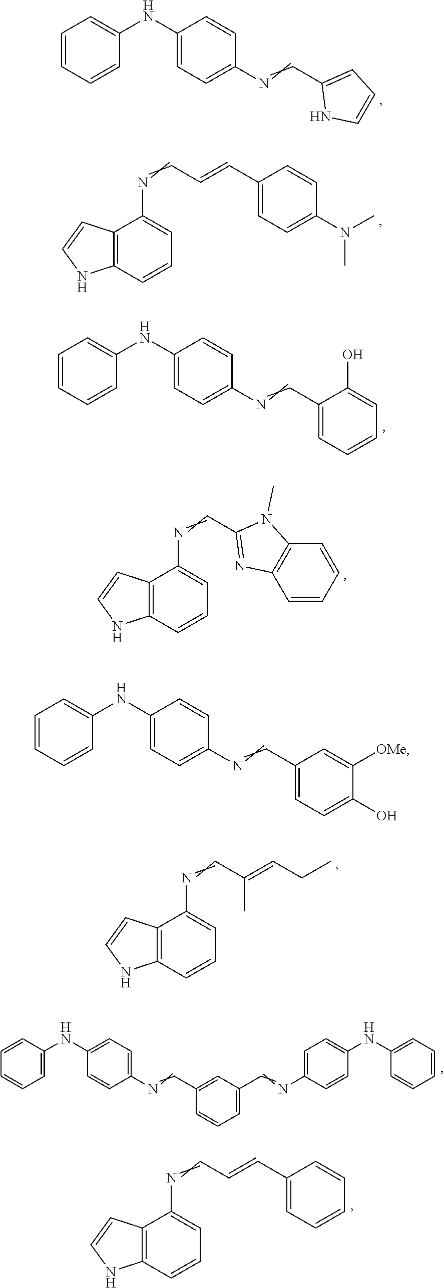

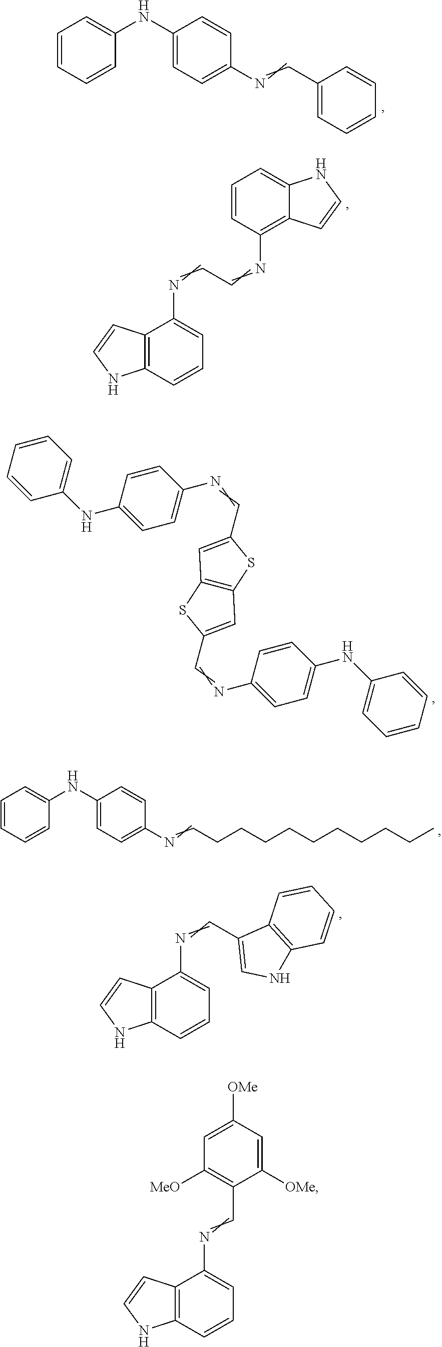

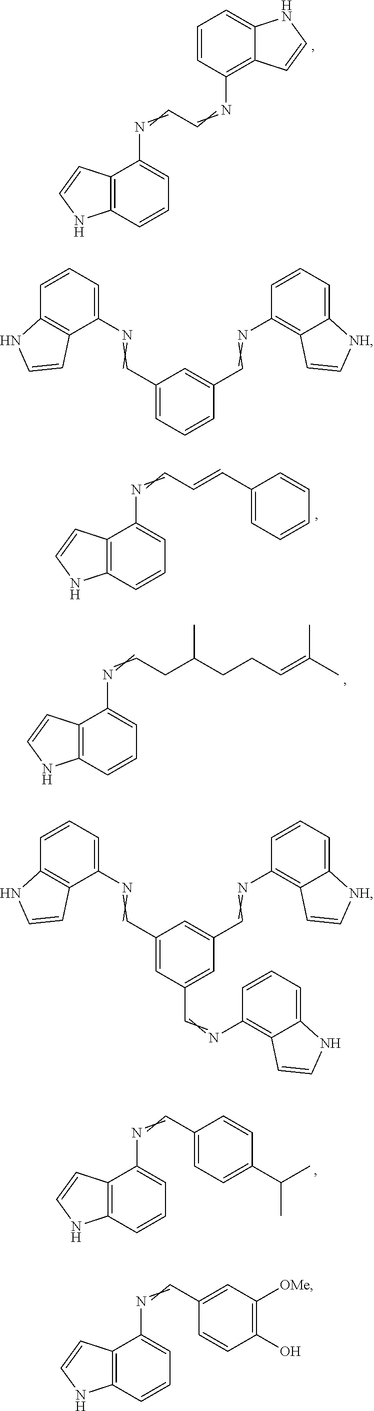

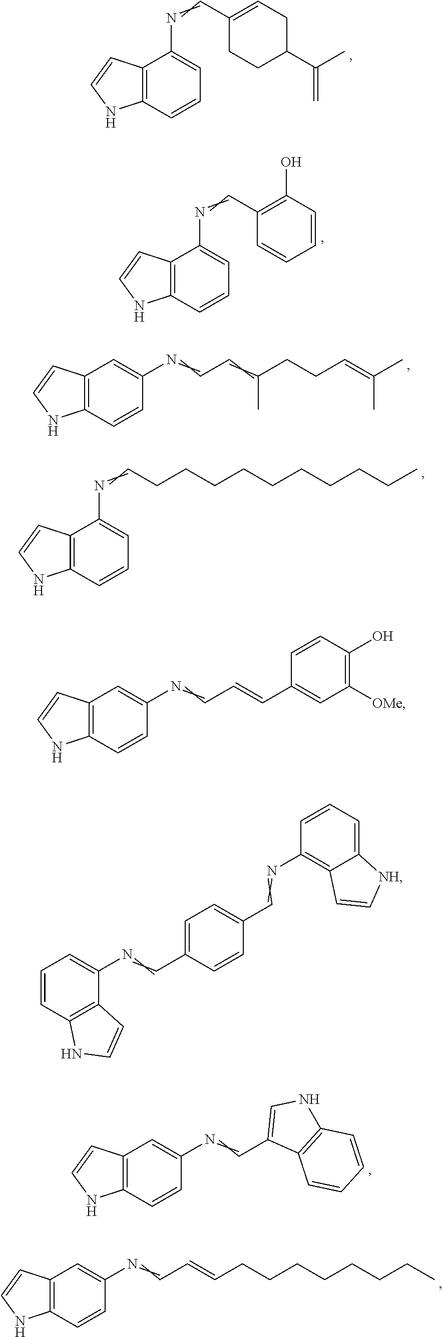

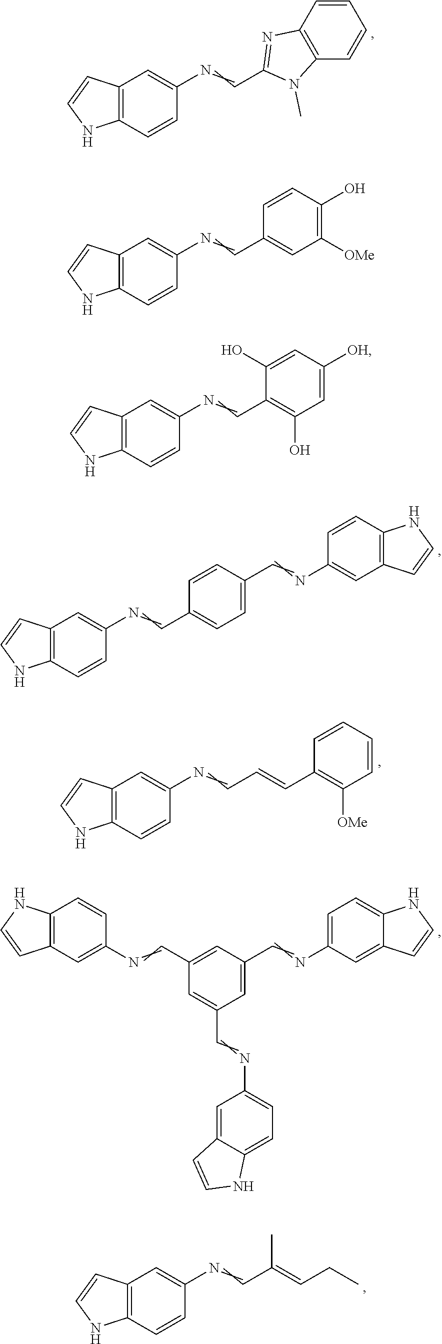

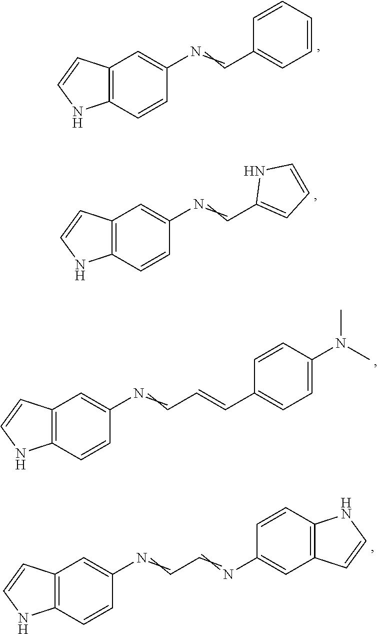 [SMILES: C(=Nc1ccc2[nH]ccc2c1)c1ccccc1 ; C(=Nc1ccc2[nH]ccc2c1)c1ccc[nH]1 ; CN(C)c1ccc(/C=C/C=Nc2ccc3[nH]ccc3c2)cc1 ; C(C=Nc1ccc2[nH]ccc2c1)=Nc1ccc2[nH]ccc2c1]

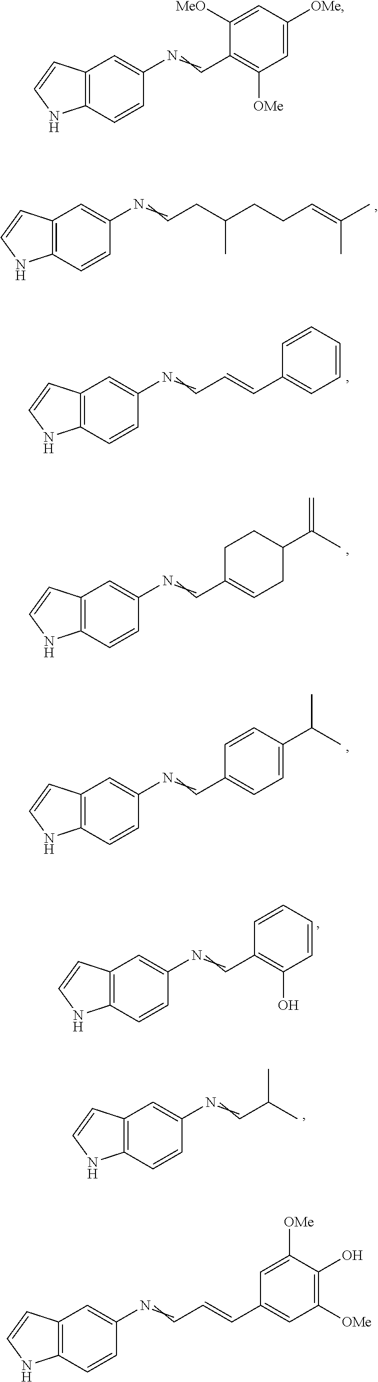

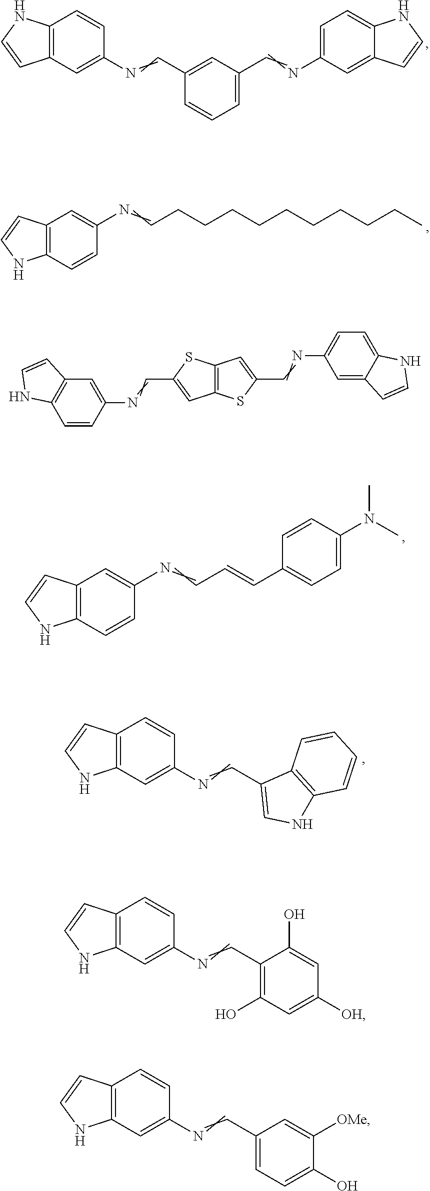

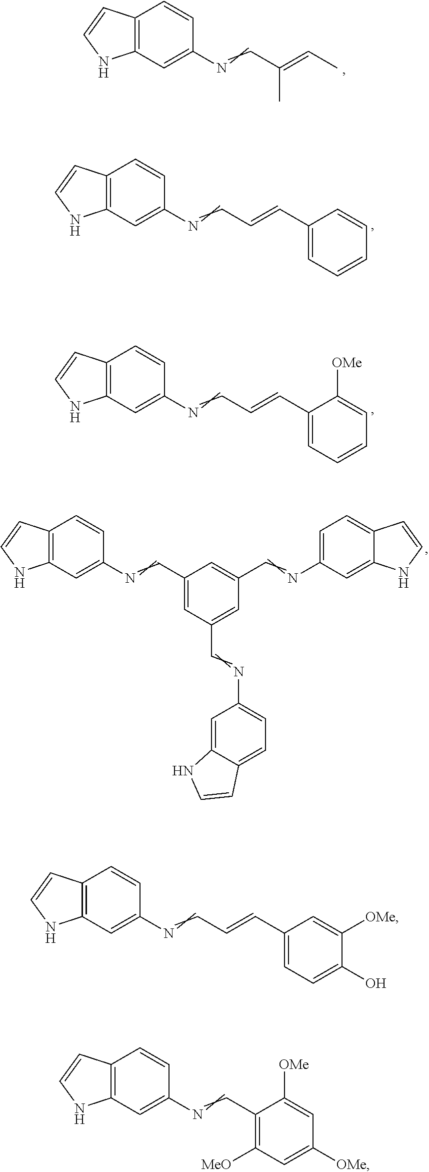 [SMILES: C/C=C(\C)C=Nc1ccc2cc[nH]c2c1 ; C(/C=C/c1ccccc1)=Nc1ccc2cc[nH]c2c1 ; COc1ccccc1/C=C/C=Nc1ccc2cc[nH]c2c1 ; C(=Nc1ccc2cc[nH]c2c1)c1cc(C=Nc2ccc3cc[nH]c3c2)cc(C=Nc2ccc3cc[nH]c3c2)c1 ; COc1cc(/C=C/C=Nc2ccc3cc[nH]c3c2)ccc1O ; COc1cc(OC)c(C=Nc2ccc3cc[nH]c3c2)c(OC)c1]

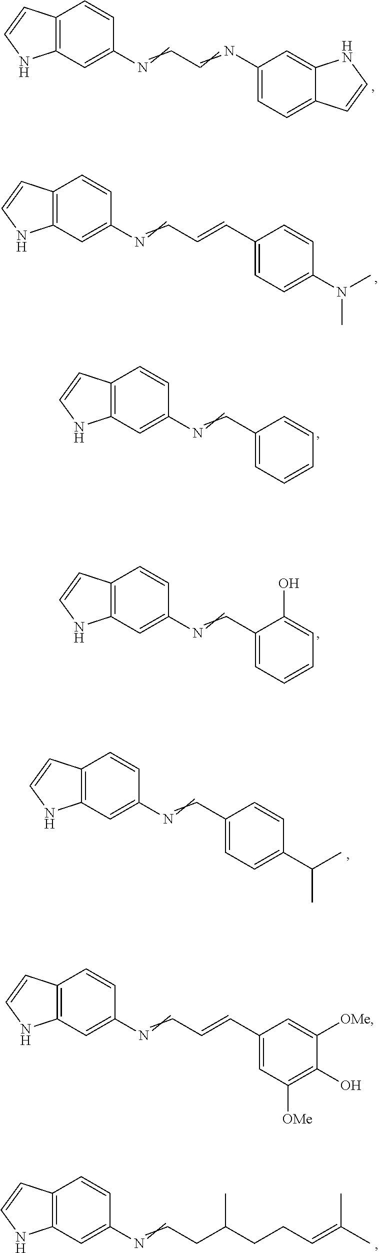

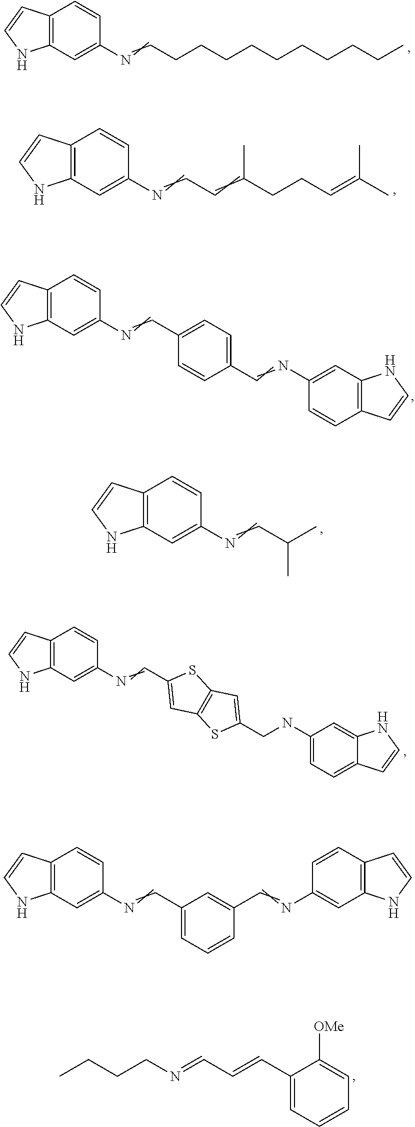

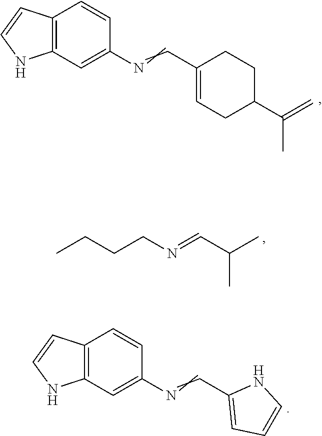

In preferred embodiments, antiozonant compounds exemplified by the compounds shown above are included in a tire composition.

Also provided herein in various embodiments are methods of treating a polymer with an effective amount of an antiozonant composition of Formula I described herein to impart a stability to said polymer from degradation by reaction with ozone. In some preferred embodiments the polymer stabilized by the antiozonant composition is rubber.

In one embodiment, a method of treating a rubber polymer to protect it against damage by ozone comprising, treating the polymer with an effective amount of a composition comprising a compound of formula:

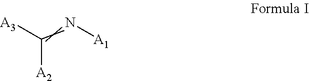

wherein A₁ is

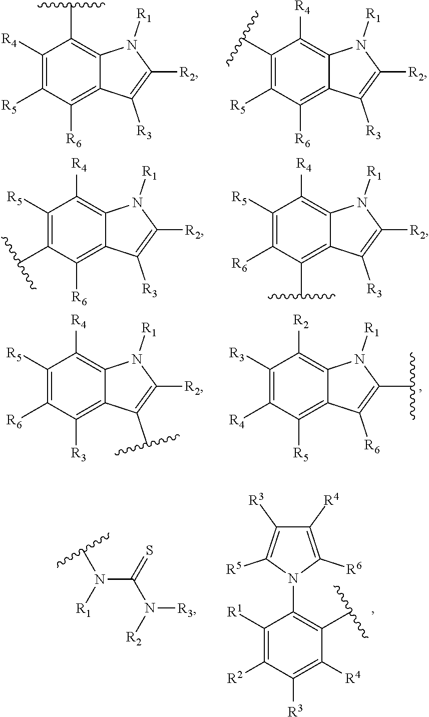

n-butyl-butyl, sec-butyl, iso-butyl, tert-butyl groups or a monocyclic heteroaryl group, each of which is optionally substituted by one or more of R1, R2, R3, R4, R5, R6,
wherein, A2 and A3 are independently selected from
a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO2, OH, NR7R8, acyl, alkyl ether or aryl ether substituents or an imine functional group bearing an A1, A2 or A3 substituent; wherein the aryl substituents are optionally substituted with one or more F, Cl, Br, I, NO2, OH, NR7R8, acyl, alkyl ether or aryl ether substituents;
wherein R1, R2, R3, R4, R5, R6 are independently selected from H, alkyl, unsaturated alkyl, F, Cl, Br, I, NO2, OH, amino, acyl, alkyl ether or aryl ether, monocyclic or polycyclic aryl or heteroaryl group;
and wherein R7, R8 are H or alkyl.

In one embodiment, the method of treating a rubber polymer to protect it against damage by ozone is described wherein the composition includes compounds which are cis or trans isomers of one or more CN imine bonds or a mixture of the isomers.

In one embodiment a rubber polymer formulation is described comprising an antiozonant compound of formula:

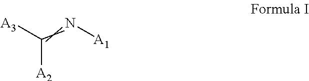

wherein A₁ is

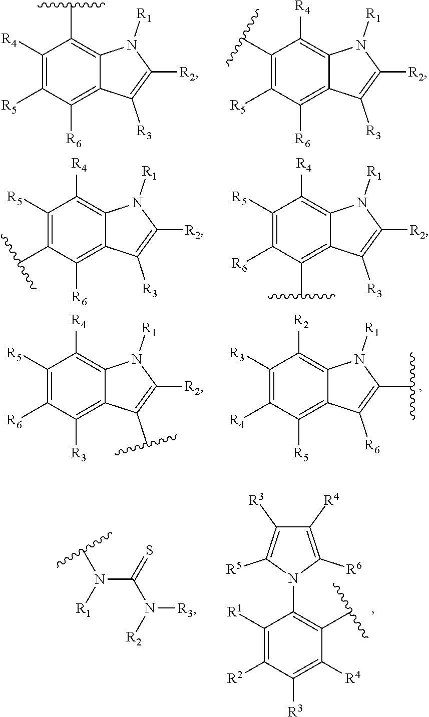

n n-butyl n-butyl, sec-butyl, iso-butyl, tert-butyl groups or a monocyclic heteroaryl group, each of which is optionally substituted by one or more of R₁, R₂, R₃, R₄, R₅, R₆,
wherein, A2 and A3 are independently selected from, a) hydrogen;
b) a saturated or unsaturated aliphatic group optionally substituted with one or more unsubstituted or substituted alkyl groups or aryl groups or an imine functional group bearing an A1, A2 or A3 substituent;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
and wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl.

In one embodiment of a rubber polymer formulation described above, the formulation includes at least one antiozonant compound of Formula I described above and the polymer in the formulation is a natural rubber.

In one embodiment of a rubber polymer formulation described above, the formulation includes at least one antiozonant compound Formula I described above and the polymer in the formulation is selected from at least one of polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber or mixtures thereof.

In one embodiment the rubber polymer formulation is a vulcanizable formulation.

In one embodiment of the rubber polymer formulation, the optionally includes one or more of additional ingredients such as tackifier resin, antioxidant, fatty acids, zinc oxide, wax, peptizer, vulcanizing agent, vulcanization accelerator, vulcanization retarder, activator, processing additive, plasticizer, pigments, and antiozonants.

In one embodiment of the rubber polymer formulation, the antiozonant compound is present in a concentration range from 0.1 to 10 parts per 100 parts by weight of rubber polymer.

In one embodiment of a rubber polymer formulation described above, the formulation includes at least one antiozonant compound of Formula I described above wherein a mixture of 1 or more antiozonant compounds is present in a total amount from 0.1 to 10 parts per 100 parts by weight of rubber polymer.

In one embodiment of a rubber polymer formulation described above, the formulation includes at least one antiozonant compound of Formula I described above wherein a mixture of 1 or more antiozonant compounds is present in the total amount of 0.1 to 5 parts per 100 parts by weight of rubber.

In one embodiment of a rubber polymer formulation described above, the formulation includes at least one antiozonant compound of Formula I described above in a vulcanized elastomeric article.

In one embodiment of a rubber polymer formulation described above, the formulation includes at least one antiozonant compound of Formula I described above in a vehicle tire.

In one embodiment, a vulcanized elastomeric article is dyed by applying an effective amount of a formulation comprising at least one compound of formula I.

In one embodiment, a composition comprising a compound of formula I is described:

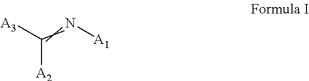

wherein A₁ is

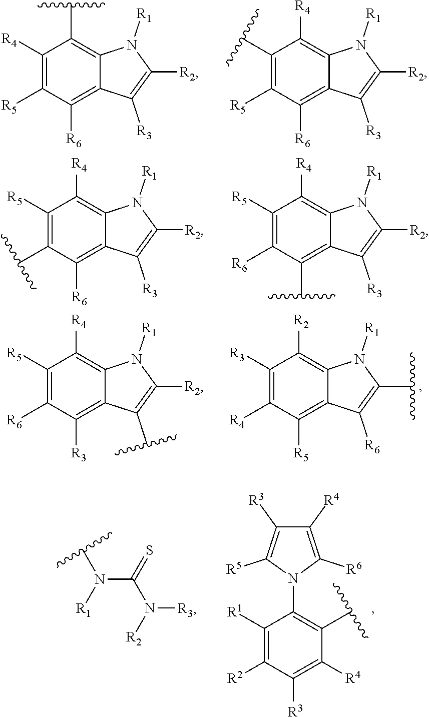

n-butyl, sec-butyl, iso-butyl, tert-butyl groups or a monocyclic heteroaryl group, optionally substituted by one or more of R₁, R₂, R₃, R₄, R₅, R₆,
wherein, A2 and A3 are independently selected from a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;

wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl.

In one embodiment, the composition comprises compounds with cis or trans isomers of a CN imine bond or a mixture of the isomers.

In other embodiments the imine functional group of formula I is reduced to a its amine derivative product molecule which is used as an antiozonant or dye or other uses.

In one embodiment, a composition comprising a compound of formula II is described:

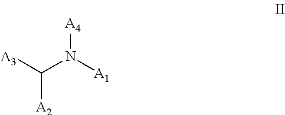

wherein A₁ is

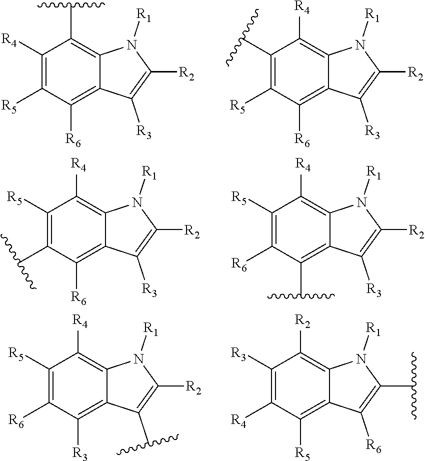

2.

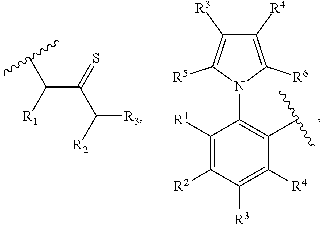

n-butyl, sec-butyl, iso-butyl, tert-butyl groups or a monocyclic heteroaryl group optionally substituted by one or more of R₁, R₂, R₃, R₄, R₅, R₆,
wherein, A2 and A3 are independently selected from
a) hydrogen;
b)

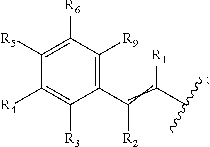

wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein A₄ is hydrogen or CHA₂A₃.

In one embodiment, a composition comprising a compound of formula III is described with formula:

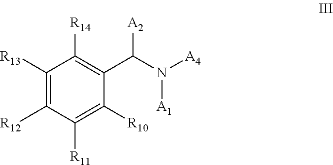

wherein A₁ is

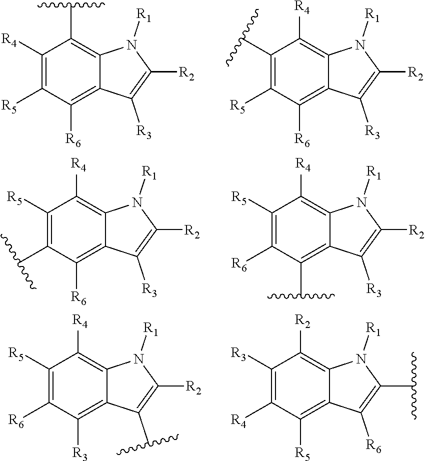

1.

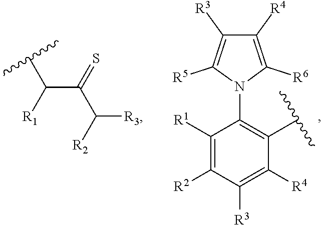

n-butyl, sec-butyl, iso-butyl, tert-butyl groups or a monocyclic heteroaryl group optionally substituted by one or more of R₁, R₂, R₃, R₄, R₅, R₆,
wherein, A2 is independently selected from
a) hydrogen;
b) a saturated or unsaturated aliphatic group optionally substituted with one or more unsubstituted or substituted alkyl groups or aryl groups or an imine functional group bearing an A1, A2 or A3 substituent;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein at least one R₁₀, R₁₁, R₁₂, R₁₃, R₁₄ is a CHA₂(NA₁A₄) group, and the remaining are independently selected from H, alkyl, unsaturated alkyl, F, Cl, Br, I, NO₂, OH, amino, acyl, alkyl ether or aryl ether, monocyclic or polycyclic aryl or heteroaryl, group; and wherein R₇, R₈ are H or alkyl,
wherein A₄ is hydrogen or CHA₂A₃.

In various embodiments, a method of treating a rubber polymer to protect it against damage by ozone is described wherein the composition includes at least one compound of formula II or formula III.

In various embodiments of a rubber polymer formulation described above, the formulation includes at least one compound of formula II or formula III described above and the polymer in the formulation is a natural rubber.

In one embodiment of a rubber polymer formulation described above, the formulation includes at least one compound of formula II or formula III described above and the polymer in the formulation is selected from at least one of polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber or mixtures thereof.

In one embodiment, at least one compound of formula II or formula III described above in the rubber polymer formulation which is a vulcanizable formulation.

In one embodiment of the rubber polymer formulation, including at least one compound of formula II or formula III described above in the formulation which optionally includes one or more of additional ingredients such as tackifier resin, antioxidant, fatty acids, zinc oxide, wax, peptizer, vulcanizing agent, vulcanization accelerator, vulcanization retarder, activator, processing additive, plasticizer, pigments, and antiozonants.

In one embodiment of the rubber polymer formulation, at least one compound of formula II or formula III is included in the formulation and the compound is present in a concentration range from 0.1 to 10 parts per 100 parts by weight of rubber polymer.

In one embodiment of a rubber polymer formulation described above, the formulation includes at least one compound of formula II or formula III described above in the formulation wherein a mixture of 1 or more compounds is present in a total amount from 0.1 to 10 parts per 100 parts by weight of rubber polymer.

In one embodiment of a rubber polymer formulation described above, the formulation includes at least one compound of formula II or formula III described above wherein a mixture of 1 or more compounds is present in the total amount of 0.1 to 5 parts per 100 parts by weight of rubber.

In one embodiment of a rubber polymer formulation described above, the formulation includes at least one compound of formula II or formula III described above in a vulcanized elastomeric article.

In one embodiment of a rubber polymer formulation described above, the formulation includes at least one compound of formula II or formula III described above in a vehicle tire. In one embodiment, a rubber polymer formulation is described wherein the formulation includes at least one antiozonant compound with a formula:

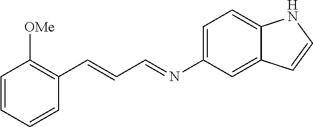 [SMILES: COc1ccccc1/C=C/C=N/c1ccc2[nH]ccc2c1]

In one embodiment, a rubber polymer formulation is described wherein the formulation includes at least one antiozonant compound with a formula:

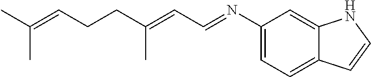 [SMILES: CC(C)=CCC/C(C)=C/C=N/c1ccc2cc[nH]c2c1]

In one embodiment, a rubber polymer formulation is described wherein the formulation includes at least one antiozonant compound with a formula:

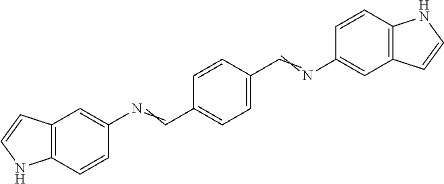 [SMILES: C(=Nc1ccc2[nH]ccc2c1)c1ccc(C=Nc2ccc3[nH]ccc3c2)cc1]

In one embodiment, a rubber polymer formulation is described wherein the formulation includes at least one antiozonant compound with a formula:

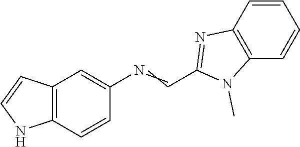 [SMILES: Cn1c(C=Nc2ccc3[nH]ccc3c2)nc2ccccc21]

In one embodiment, a rubber polymer formulation is described wherein the formulation includes at least one antiozonant compound with a formula:

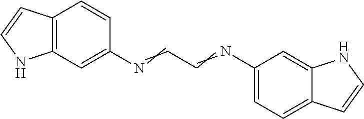 [SMILES: C(C=Nc1ccc2cc[nH]c2c1)=Nc1ccc2cc[nH]c2c1]

In one embodiment, a rubber polymer formulation is described wherein the formulation includes at least one antiozonant compound with a formula:

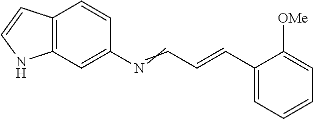 [SMILES: COc1ccccc1/C=C/C=Nc1ccc2cc[nH]c2c1]

In one embodiment, a rubber polymer formulation is described wherein the formulation includes at least one antiozonant compound with a formula:

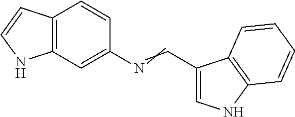 [SMILES: C(=Nc1ccc2cc[nH]c2c1)c1c[nH]c2ccccc12]

In one embodiment, a rubber polymer formulation is described wherein the formulation includes at least one antiozonant compound with a formula:

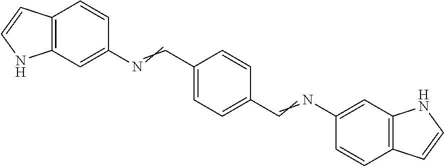 [SMILES: C(=Nc1ccc2cc[nH]c2c1)c1ccc(C=Nc2ccc3cc[nH]c3c2)cc1]

In one embodiment, a rubber polymer formulation is described wherein the formulation includes at least one antiozonant compound with a formula:

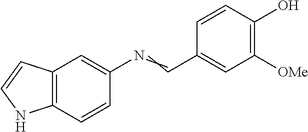 [SMILES: COc1cc(C=Nc2ccc3[nH]ccc3c2)ccc1O]

In one embodiment, a rubber polymer formulation is described wherein the formulation includes at least one antiozonant compound with a formula:

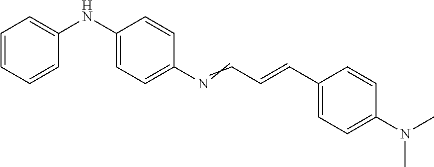 [SMILES: CN(C)c1ccc(/C=C/C=Nc2ccc(Nc3ccccc3)cc2)cc1]

In one embodiment, a rubber polymer formulation is described wherein the formulation includes at least one antiozonant compound with a formula:

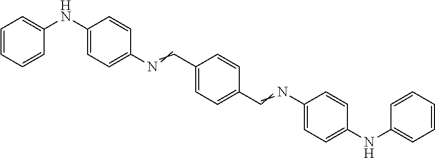 [SMILES: C(=Nc1ccc(Nc2ccccc2)cc1)c1ccc(C=Nc2ccc(Nc3ccccc3)cc2)cc1]

In one embodiment, a rubber polymer formulation is described wherein the formulation includes at least one antiozonant compound with a formula:

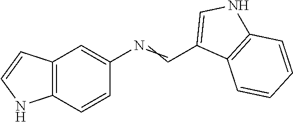 [SMILES: C(=Nc1ccc2[nH]ccc2c1)c1c[nH]c2ccccc12]

In one embodiment, a rubber polymer formulation is described wherein the formulation includes at least one antiozonant compound with a formula:

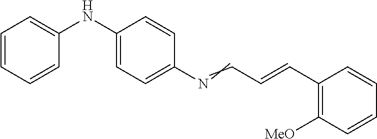 [SMILES: COc1ccccc1/C=C/C=Nc1ccc(Nc2ccccc2)cc1]

In one embodiment, a rubber polymer formulation is described wherein the formulation includes at least one antiozonant compound with a formula:

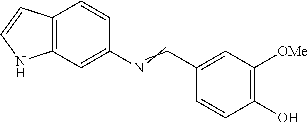 [SMILES: COc1cc(C=Nc2ccc3cc[nH]c3c2)ccc1O]

In one embodiment, a rubber polymer formulation is described wherein the formulation includes at least one antiozonant compound with a formula:

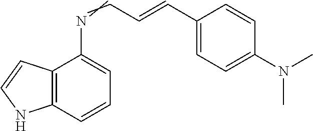 [SMILES: CN(C)c1ccc(/C=C/C=Nc2cccc3[nH]ccc23)cc1]

In one embodiment, a rubber polymer formulation is described wherein the formulation includes at least one antiozonant compound with a formula:

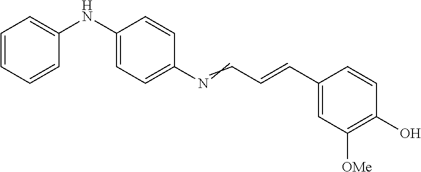 [SMILES: COc1cc(/C=C/C=Nc2ccc(Nc3ccccc3)cc2)ccc1O]

In one embodiment, a rubber polymer formulation is described wherein the formulation includes at least one antiozonant compound with a formula:

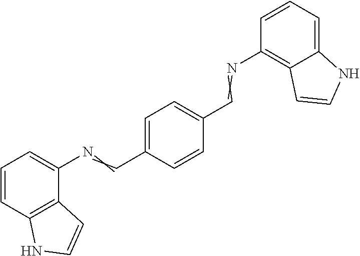 [SMILES: C(=Nc1cccc2[nH]ccc12)c1ccc(C=Nc2cccc3[nH]ccc23)cc1]

In one embodiment, a rubber polymer formulation is described wherein the formulation includes at least one antiozonant compound with a formula:

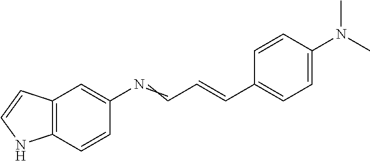 [SMILES: CN(C)c1ccc(/C=C/C=Nc2ccc3[nH]ccc3c2)cc1]

In one embodiment, a rubber polymer formulation is described wherein the formulation includes at least one antiozonant compound with a formula:

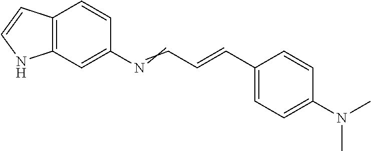 [SMILES: CN(C)c1ccc(/C=C/C=Nc2ccc3cc[nH]c3c2)cc1]

In one embodiment is a method of treating a rubber polymer to protect it against damage by ozone comprising, treating the polymer with an effective amount of a composition comprising a compound of formula:

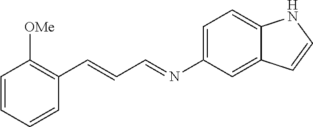 [SMILES: COc1ccccc1/C=C/C=N/c1ccc2[nH]ccc2c1]

In one embodiment is a method of treating a rubber polymer to protect it against damage by ozone comprising, treating the polymer with an effective amount of a composition comprising a compound of formula:

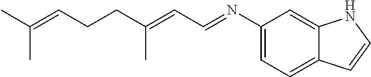 [SMILES: CC(C)=CCC/C(C)=C/C=N/c1ccc2cc[nH]c2c1]

In one embodiment is a method of treating a rubber polymer to protect it against damage by ozone comprising, treating the polymer with an effective amount of a composition comprising a compound of formula:

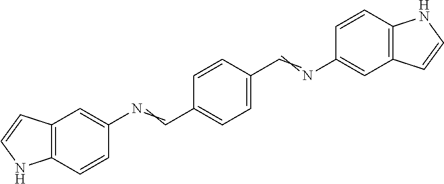 [SMILES: C(=Nc1ccc2[nH]ccc2c1)c1ccc(C=Nc2ccc3[nH]ccc3c2)cc1]

In one embodiment is a method of treating a rubber polymer to protect it against damage by ozone comprising, treating the polymer with an effective amount of a composition comprising a compound of formula:

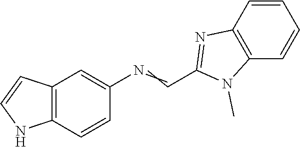 [SMILES: Cn1c(C=Nc2ccc3[nH]ccc3c2)nc2ccccc21]

In one embodiment is a method of treating a rubber polymer to protect it against damage by ozone comprising, treating the polymer with an effective amount of a composition comprising a compound of formula:

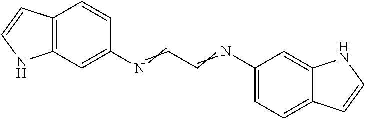 [SMILES: C(C=Nc1ccc2cc[nH]c2c1)=Nc1ccc2cc[nH]c2c1]

In one embodiment is a method of treating a rubber polymer to protect it against damage by ozone comprising, treating the polymer with an effective amount of a composition comprising a compound of formula:

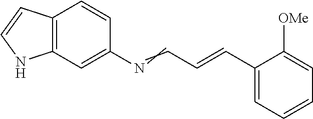 [SMILES: COc1ccccc1/C=C/C=Nc1ccc2cc[nH]c2c1]

In one embodiment is a method of treating a rubber polymer to protect it against damage by ozone comprising, treating the polymer with an effective amount of a composition comprising a compound of formula:

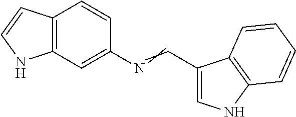 [SMILES: C(=Nc1ccc2cc[nH]c2c1)c1c[nH]c2ccccc12]

In one embodiment is a method of treating a rubber polymer to protect it against damage by ozone comprising, treating the polymer with an effective amount of a composition comprising a compound of formula:

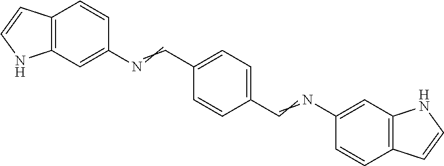 [SMILES: C(=Nc1ccc2cc[nH]c2c1)c1ccc(C=Nc2ccc3cc[nH]c3c2)cc1]

In one embodiment is a method of treating a rubber polymer to protect it against damage by ozone comprising, treating the polymer with an effective amount of a composition comprising a compound of formula:

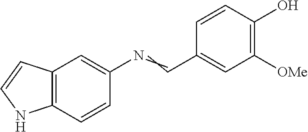 [SMILES: COc1cc(C=Nc2ccc3[nH]ccc3c2)ccc1O]

In one embodiment is a method of treating a rubber polymer to protect it against damage by ozone comprising, treating the polymer with an effective amount of a composition comprising a compound of formula:

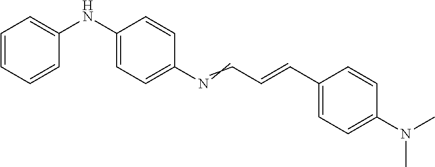 [SMILES: CN(C)c1ccc(/C=C/C=Nc2ccc(Nc3ccccc3)cc2)cc1]

In one embodiment is a method of treating a rubber polymer to protect it against damage by ozone comprising, treating the polymer with an effective amount of a composition comprising a compound of formula:

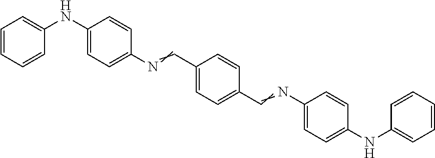 [SMILES: C(=Nc1ccc(Nc2ccccc2)cc1)c1ccc(C=Nc2ccc(Nc3ccccc3)cc2)cc1]

In one embodiment is a method of treating a rubber polymer to protect it against damage by ozone comprising, treating the polymer with an effective amount of a composition comprising a compound of formula:

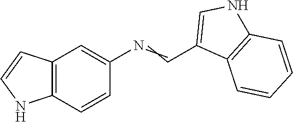 [SMILES: C(=Nc1ccc2[nH]ccc2c1)c1c[nH]c2ccccc12]

In one embodiment is a method of treating a rubber polymer to protect it against damage by ozone comprising, treating the polymer with an effective amount of a composition comprising a compound of formula:

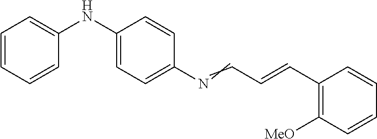 [SMILES: COc1ccccc1/C=C/C=Nc1ccc(Nc2ccccc2)cc1]

In one embodiment is a method of treating a rubber polymer to protect it against damage by ozone comprising, treating the polymer with an effective amount of a composition comprising a compound of formula:

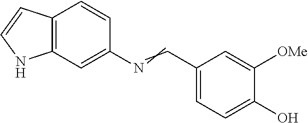 [SMILES: COc1cc(C=Nc2ccc3cc[nH]c3c2)ccc1O]

In one embodiment is a method of treating a rubber polymer to protect it against damage by ozone comprising, treating the polymer with an effective amount of a composition comprising a compound of formula:

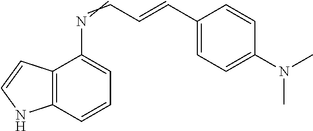 [SMILES: CN(C)c1ccc(/C=C/C=Nc2cccc3[nH]ccc23)cc1]

In one embodiment is a method of treating a rubber polymer to protect it against damage by ozone comprising, treating the polymer with an effective amount of a composition comprising a compound of formula:

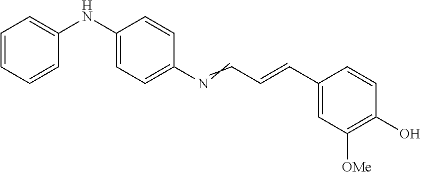 [SMILES: COc1cc(/C=C/C=Nc2ccc(Nc3ccccc3)cc2)ccc1O]

In one embodiment is a method of treating a rubber polymer to protect it against damage by ozone comprising, treating the polymer with an effective amount of a composition comprising a compound of formula:

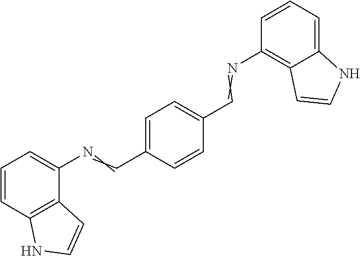 [SMILES: C(=Nc1cccc2[nH]ccc12)c1ccc(C=Nc2cccc3[nH]ccc23)cc1]

In one embodiment is a method of treating a rubber polymer to protect it against damage by ozone comprising, treating the polymer with an effective amount of a composition comprising a compound of formula:

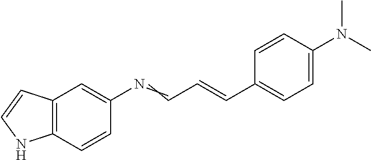 [SMILES: CN(C)c1ccc(/C=C/C=Nc2ccc3[nH]ccc3c2)cc1]

In one embodiment is a method of treating a rubber polymer to protect it against damage by ozone comprising, treating the polymer with an effective amount of a composition comprising a compound of formula:

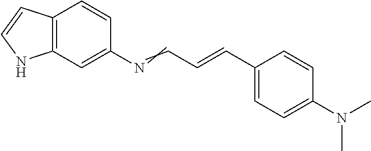 [SMILES: CN(C)c1ccc(/C=C/C=Nc2ccc3cc[nH]c3c2)cc1]

Also provided herein in various embodiments are methods of synthesizing antiozonant compositions.

The term “rubber” includes natural or synthetic elastomers, including polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber, and the like, and mixtures thereof.

The rubber matrix when used in tires, hoses, power transmission belts and other industrial products has good compatibility with fillers, such as carbon black and silica. The rubbery matrix can be functionalized with various compounds to make a rubber polymer formulation, such as tackifier resin, antioxidant, fatty acids, zinc oxide, wax, peptizer, vulcanizing agent, vulcanization accelerator, vulcanization retarder, activator, processing additive, plasticizer, pigments, and antiozonants. Rubber polymer formulations used in industry to manufacture various items exemplified by vulcanized rubber items, tires for example are well known and can incorporate one or more compounds described in the various embodiments described herein including compounds of formula I and their reduced imine derivatives.

The term functional group refers to an atom or a group of atoms in a molecule with distinctive chemical properties, regardless of the other atoms in the molecule. The atoms in a functional group are linked to each other and to the rest of the molecule by covalent bonds. Examples of functional groups include ester, ether, OH, COOH, NO₂, NH₂, Cl, Br, etc.

The term antiozonant describes a compound that is added to any material such as a polymer or another compound or mixture of compounds or polymers or both at any time during its lifetime or during manufacturing to protect it against damage from ozone. The damage can be caused by oxidation reactions with ozone or oxidation products produced or ozone. Attrition of the molecular structure of a polymer (exemplified by rubber polymer) can lead to diminished performance of the polymer for its intended use. Examples of sign of damage might include fine cracking on the surface of rubber in stretched areas commonly called “dry rot”, becoming stiff, hard, and losing its elasticity, leading to a loss of tensile strength and eventual failure.

The term monocyclic or polycyclic refers to organic molecules with ring structures, differing in the number of rings: monocyclic (like benzene) have one ring, while polycyclic (like naphthalene, anthracene) have two or more fused or linked rings.

Aryl refers to compounds having aromatic rings made only of carbon and hydrogen (like benzene), while heteroaryl compounds are aromatic rings where one or more carbons are replaced by a heteroatom (like Nitrogen, Oxygen, or Sulfur), making them heterocyclic aromatics (e.g., pyridine, furan). Both feature stable, cyclic structures with delocalized electrons, but aryl groups are purely hydrocarbon-based, whereas heteroaryls incorporate non-carbon atoms.

The term substituted refers to organic molecules where one or more atoms (usually hydrogen) on a parent structure, like a hydrocarbon chain or ring, are replaced by different atoms or groups of atoms (substituents), changing the molecule's structure and properties.

The term “effective amount” of a composition, compound or property as provided herein is meant such amount as is capable of performing the function of the compound or property for which an effective amount is expressed. As is pointed out herein, the exact amount required will vary from process to process, depending on recognized variables such as the compounds employed and various internal and external conditions observed as would be interpreted by one of ordinary skill in the art. Thus, it is not possible to specify an exact “effective amount,” though preferred ranges have been provided herein. An appropriate effective amount may be determined, however, by one of ordinary skill in the art using only routine experimentation.

The term “optional” or “optionally” means that the subsequently described event or circumstance may or may not occur, and that the description includes instances in which said event or circumstance occurs and instances where it does not. For example, the phrase “optionally comprising a defoaming agent” means that the composition may or may not contain a defoaming agent and that this description includes compositions that contain and do not contain a foaming agent.

As used herein, the term “independently selected from” refers to the recited groups being the same, different, or mixtures thereof, unless the context clearly dictates otherwise. Thus, in this definition, the phrase “R₁, R₂, and R₃ are independently selected from Fl, Cl, Br, or I” includes, for example, when R₁, R₂, and R₃ are all the same (for example all R₁, R₂, and R₃ are bromine atoms “Br”), when R₁, R₂, and R₃ are all different (i.e. when they are Cl, I and Br respectively for example), or when R₁, and R₂ are the same but R₃ is different, and other similar permutations.

Imine isomers refers to imines compounds which exhibit cis-trans isomerism (also called E/Z isomerism) because of restricted rotation around the carbon-nitrogen double bond (C═N). Like alkenes, substituents on the imine's carbon and nitrogen atoms can be on the same side (cis) or opposite sides (trans) of the C═N bond, creating distinct geometric isomers that differ in spatial arrangement.

The term “substantially pure” refers to a formulation that is at least about 90% (e.g., at least 90%) in purity weight/weight of a total composition. In a more preferred embodiment, the purity is at least about 95% (e.g., at least about 95%) weight-to-weight, or at least about 98% (e.g., at least about 98%) purity.

The typical loadings of the antiozonants may vary as needed for the rubber application, severity of use, environmental and other factors. For the several embodiments disclosed, the loading of antiozonants described herein is between 0.2 and 20 phr, 0.2 and 15 phr, alternatively between 0.5 and 10 phr, and between 1 and 5 phr, 0.1 to 5 phr, or at least 0.3 phr, or at least 1 phr, or at least 2 phr. The term “phr” refers to parts of a respective material per 100 parts by weight of rubber, or elastomer. As used herein, the term “about” is defined as plus or minus ten percent of a recited value. For example, about 1.0 g means 0.9 g to 1.1 g.

The amounts, percentages, and ranges disclosed herein are not meant to be limiting, and increments between the recited amounts, percentages, and ranges are specifically envisioned as part of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

It will be obvious to those skilled in the art that such embodiments are provided by way of example only. Numerous variations, changes, and substitutions will occur to those skilled in the art without departing from the embodiments of the claims. Various alternatives to the embodiments of the claims described herein may be employed in practicing the use of compositions and methods of treatment described herein. It is intended that the included claims define the scope of the various compositions and methods of treatment described herein and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

The term “consisting essentially of” excludes additional method (or process) steps or composition components that substantially interfere with the intended activity of the method (or process) or composition. This term may be substituted for inclusive terms such as “comprising” or “including” to more narrowly define any of the disclosed embodiments or combinations/sub-combinations thereof. Furthermore, the exclusive term “consisting” is also understood to be substitutable for these inclusive terms.

Described below are abbreviations used herein.

phr The term “phr” refers to parts of a respective material per 100 parts by weight of rubber, or elastomer.

GNR Guayule natural rubber

SB Schiff base, a Schiff base is an organic compound featuring a carbon-nitrogen double bond (C═N), specifically an imine formed from the condensation of a primary amine with an aldehyde or ketone

RID refractive index detector

6PPD p-phenylenediamines

6PPQ-6PPD-quinone or 6PPD-Q; quinone oxidation product of 6PPD; for example, 2-anilino-5-(4-methylpentan-2-ylamino)cyclohexa-2,5-diene-1,4-dione.-,

### Examples

Having now generally described the compositions, methods of treatment and other embodiments described herein, the same will be better understood by reference to certain specific examples, which are included herein only to further illustrate the embodiments and are not intended to limit the scope of the same as defined by the claims.

Synthesis of Schiff bases. Schiff bases were synthesized through the condensation of aromatic amines (examples in Table 2) with a variety of aldehydes (examples in Table 1). All solvents and reagents were purchased from suppliers (Thermo Scientific, Sigma-Aldrich, Ambeed) and used without further purification. In a 50 mL round-bottom flask with stir bar, 5-aminoindole (0.500 g, 3.78 mmol) and 2-methoxy-cinnamaldehyde (0.613 g, 3.78 mmol, 1 equiv.) were dissolved in EtOH (200 proof, 10 mL) with molecular sieves. Acetic acid (21 μL, 0.38 mmol, 0.1 equiv.) was then added to the flask. The reaction mixture was stirred at room temperature for 2 hours. The resulting solid was vacuum filtered through a Hirsch funnel and washed with additional EtOH. Trace solvent was removed from the resulting powder and dried under vacuum using a rotary evaporator (yield: 0.511 g, 48.9%). ¹H NMR (80 MHz, DMSO-d₆) δ 11.19 (bs, 1H), 8.46 (dd, 1H), 7.44 (m, 11H), 6.45 (bs, 1H), 3.87 (s, 3H).

Characterization of Schiff bases. ¹H-NMR spectra were recorded on a 80 MHz Magritek Spinsolve 80 NMR spectrometer. Deuterated DMSO (DMSO-d₆), chloroform (CDCl₃), or ethanol (EtOH-d₆) were used as solvents for all NMR spectra. Thermal properties of SBs were determined through thermogravimetric analysis (TGA) using

Screening of Schiff bases. The antiozonant activity of candidate molecules was evaluated using solution viscometry SBs were dissolved in 20.0 g/L solutions of Guayule natural rubber (GNR, Yulex Corporation, United States) in tetrahydrofuran (THF) to achieve an antiozonant concentration of 10 parts per hundred rubber (phr). The initial viscosity of a 50 mL aliquot of this solution was evaluated using a Brookfield LVDVE Viscometer (United States) at 100 rpm. Ozone (O₃) was generated using a 2G Lab Benchtop Ozone Generator (A2Z Ozone, United States), and bubbled through the stabilized rubber solution in 60 s intervals for a total of 6 min. The viscosity of the solution was determined after each exposure to O₃ to track GNR degradation throughout the experiment.

Schiff bases for various amine-aldehyde combinations between Tables 2 and 3 were synthesized according to the described procedure and characterized accordingly. The disappearance of aldehyde C—H resonances (δ≈9-10 ppm) and corresponding appearance of imine C—H resonances in ¹H-NMR (δ≈8-9 ppm) were used to monitor the completion of Schiff base condensations.

Example 1. The reaction of 5-aminoindole (B) with 2-methoxycinnamladehyde (6) to form N-(1H-indol-5-yl)-3-(2-methoxyphenyl) prop-2-en-1-imine (B6) can be seen in FIG. 3. In a 50 mL round-bottom flask with stir bar, 5-aminoindole (0.500 g, 3.78 mmol) and 2-methoxy-cinnamaldehyde (0.613 g, 3.78 mmol, 1 equiv.) were dissolved in EtOH (200 proof, 10 mL) with molecular sieves Acetic acid (21 μL, 0.38 mmol, 0.1 equiv.) was then added to the flask. The reaction mixture was stirred at room temperature for 2 hours. The resulting solid was vacuum filtered through a Hirsch funnel and washed with additional EtOH. Trace solvent was removed from the resulting powder and dried under vacuum using a rotary evaporator (yield: 0.511 g, 48.9%). ¹H NMR (80 MHz, DMSO-d₆): δ 11.19 (bs, 1H), 8.46 (dd, 1H), 7.44 (m, 11H), 6.45 (bs, 1H), 3.87 (s, 3H). FTIR (cm⁻¹): 2600-3200 (broad), 1617 (sharp), 1560 (sharp).

Upon quenching of reactions and isolation of products, spectral characterization confirmed the desired structure was obtained. The ¹H-NMR resonance at 8.46 (d, 1H) corresponds to the HC═N proton, where the presence of E and Z stereoisomers of B6 account for the doublet multiplicity. Likewise, the appearance of sharp peaks in the FTIR spectrum at 1617 and 1560 cm⁻¹, which are due to C═N stretching, confirm the presence of an imine bond in the desired structure.

Example 2. The reaction of 6-aminoindole (C) with citral (3) to form N-(1H-indol-6-yl)-3,7-dimethylocta-2,6-dien-1-imine (C3) can be seen in Scheme 21H NMR (80 MHz, DMSO-d₆): 11.01 (broad s, 1H), 10.39 (s, 0.2H), 8.48 (dd, 1.11H), 7.45 (dd, 1.22H), 7.26 (m, 1.24H), 7.13 (m, 1.37H), 6.89 (m, 1.44H), 6.52 (m, 0.26H), 6.36 (m, 1.44H), 6.11 (d, 1.36H), 5.10 (m, 1.51H), 4.63 (broad, 0.44H).

Here, the ¹H-NMR resonance at 8.48 (dd), as well as the absence of an aldehyde C—H resonance, indicates the formation of the C═N bond upon the condensation of C and 3 to form C3. Residual peaks at 10.39 (s) and 6.52 (m) indicate the presence of unreacted C in the reaction mixture, and a product purity of approximately 80%.

5-aminoindole (0.137 mol) and 2-methoxycinnamaldehyde (0.095 mol) were mixed in 50 mL of ethanol at room temperature. After 30 mins, an orange precipitation was formed. The compounds were combined and mixed for 5 hours at room temperature. The solid was collected by vacuum filtration.

### Reduction Procedure:

The Schiff Base (0.108 mol) was dissolved in 25 mL of 30 methanol: 70 chloroform at 0° C. NaBH₄ (0.173 mol) was dissolved in 20 mL methanol at 0° C. When the bubbling of the NaBH₄ solution decreased, it was added to the Schiff Base solution. The mixture removed from the ice bath after 60 mins and left at room temperature for 24 hours. The solution changed from orange to white. The precipitation was vacuum-filtered.

NaBH₄ was found the most efficient reducing agent without producing side products. NaBH₄ dissolves and has the best reactivity in methanol. Dissolve NaBH₄ in methanol first and then add it to the reaction flask. If there is no solid form, the reaction solution is washed with sodium bicarbonate. Chloroform works better than ethyl acetate to separate the organic and inorganic layers. The organic layer is dried by MgSO₄, and solvent is removed by rotary evaporator.

### EXAMPLE: Concentration Dependance of Antiozonant Activity

The antiozonant activity of Schiff base of 6-aminoindole and citral was evaluated at two concentrations using solution viscometry [0156]. The Schiff base (C3) was prepared as described [0053] and was dissolved in 20.0 g/L solutions of Guayule natural rubber in tetrahydrofuran (THF) to achieve an antiozonant concentration of 10 parts per hundred rubber (phr) (1×) or 20 parts per hundred rubber (2×). The initial viscosity of a 50 mL aliquot of this solution was evaluated using a Brookfield LVDVE Viscometer (United States) at 100 rpm. Ozone (O₃) was generated using a 2G Lab Benchtop Ozone Generator (A2Z Ozone, United States) and bubbled through the unstabilized (Blank) and stabilized rubber solutions of 6PPD, and SB at 1 or 2 equimolar concentrations (1×, 2×). The viscosity of the solution was determined after each 60s exposure to O₃ to track GNR degradation throughout the 6-minute experiment. The SB at 2× concentration protected the rubber in solution at equal to better levels than 6PPD.

### Example IN-RUBBER PERFORMANCE

Molecules B6, B21, and C2 were mixed into a model rubber compound similar to a tire sidewall formulation using standard rubber mixing methods. All AOs were added on an equivalent molar basis. A scorch test (similar to ASTM D1646) was used to determine the process safety window, expressed as time to increase torque by 5 Mooney Units (MU) or 35 MU. Molecule B21 had similar scorch behavior to 6PPD; molecules B6 and C2 had shorter times, i.e., less process safety under the conditions tested and may require cure system reformulation.

Rubber sheets were prepared by press curing and die cutting, into specimens for ozone resistance testing using conditions similar to ASTM D1149. The modulus (stiffness) of the compound was measured before (Fo) and after (F) exposure to ozone under static (constant stress) and dynamic (stress cycles) conditions. Molecules B6 and B21 showed force retention from 80 to 108% of that found for the 6PPD compound. C2 showed lower force retention, about 75% of the 6PPD compound performance, suggesting higher molar equivalents would be needed.

| Sidewall | B6 (Ex#1) | B21 | C2 (Ex#2) | 6PPD |
|---|---|---|---|---|
| T5 (min) | 12 | 23 | 3 | 20 |
| T35 (min) | 14 | 25 | 4 | 24 |
| F/Fo | 96 | 91 | 67 | 89 |
| (static, | | | | |
| 96 h) | | | | |
| F/Fo | 94 | 84 | 78 | 103 |
| (dynamic, | | | | |
| 96 h) | | | | |

Demonstration of antiozonant activity. Antiozonant activity of Schiff bases was determined using solution ozonation and viscometry as described above. Absolute viscosities throughout the experiment were used to determine

$${η}_{t}^{α} ,$$

the viscosity ratio of solution α at time t according to

$${η}_{t}^{α} = {v}_{t}^{α} / {v}_{0}^{α} , \qquad [ Eq . 1 ]$$

where

$${v}_{t}^{α}$$

is the absolute viscosity of solution α at time t and

$${v}_{0}^{α}$$

is the absolute viscosity of solution α at time t=0 s. Values of

$${η}_{t = 120}^{α}$$

are reported in Table 4.

From these, antiozonant percent efficiencies

$$( {φ}_{t}^{α} )$$

for each SB were computed as

$${φ}_{t}^{α} = \frac{{η}_{t}^{α} - {η}_{t}^{blank}}{{η}_{t}^{α} - {η}_{t}^{6 PPD}} , \qquad [ Eq . 2 ]$$

where

$${η}_{t}^{blank} and {η}_{t}^{6 PPD}$$

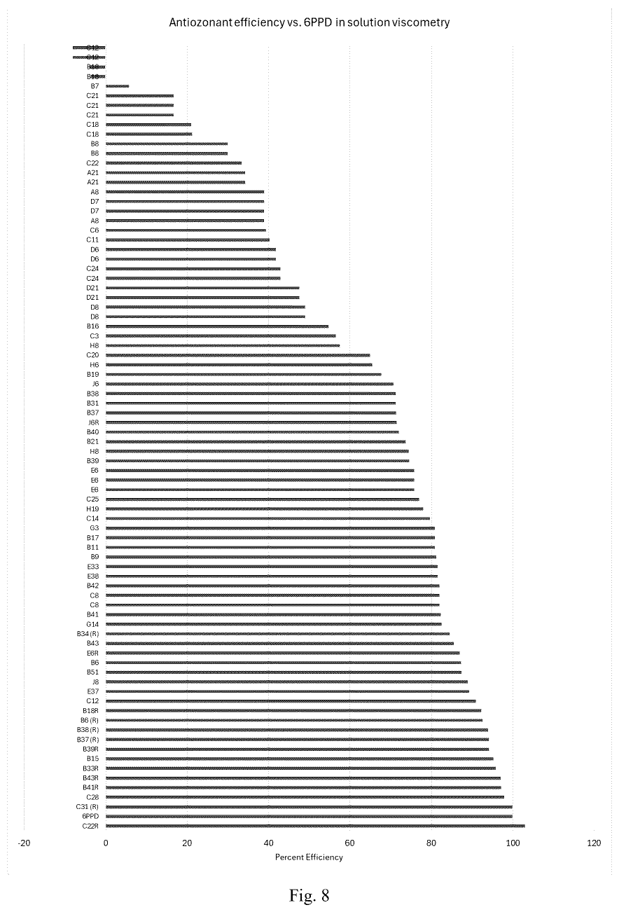

and are the viscosity ratios for blank and 6PPD solutions at time t, respectively. Percent efficiencies as determined by Eq. 2 define a scale of antiozonant performance where 0% corresponds to the baseline performance of an untreated GNR blank, and 100% corresponds to the performance of 6PPD. This scale provides an intuitive, normalized scale for describing antiozonant performance than viscosity ratios (Eq. 1) alone. These values are presented in both Table 4 and FIG. 8.

Percent efficiencies for SBs range from −8.0 to 103% of the antiozonant performance of 6PPD (Table 4, FIG. 8). The product C28 demonstrates rubber protection upwards of 98% of that of 6PPD when added in equimolar concentrations to rubber solutions. Additionally, the coverage of the normalized efficiency scale seen in FIG. 8 demonstrates the possibility of tuning SB properties to achieve the desired antiozonant performance, and the results herein do not necessarily represent the upper end of the performance range for this diverse class of molecules.

Percent efficiency of exemplified Schiff base antiozonants as determined through solution ozonation of Guayule natural rubber shown in table 4 below.

TABLE 4

| Code | Amine | Aldehyde | ${η}_{t = 120}^{α}$ | ${φ}_{t = 120}^{α}$ |
|---|---|---|---|---|
| B6 | 5-aminoindole | 2-methoxycinnamaldehyde | 0.857 | 87.3% |
| B21 | 5-aminoindole | Terephthalaldehyde | 0.823 | 73.7% |
| C3 | 6-aminoindole | Citral | 0.796 | 56.6% |
| B16 | 5-aminoindole | 1-methyl-1H-benzimidazole-2-carbaldehyde | 0.792 | 54.8% |
| C24 | 6-aminoindole | Glyoxal | 0.768 | 42.6% |
| C6 | 6-aminoindole | 2-methoxycinnamaldehyde | 0.761 | 39.4% |
| C | 6-aminoindole | Indole-3-carboxaldehyde | 0.725 | 21.3% |
| C21 | 6-aminoindole | Terephthalaldehyde | 0.716 | 16.7% |
| B | 5-aminoindole | 4-hydroxy-3-methoxycinnamaldehyde | 0.694 | 5.8% |
| A | 4-aminodiphenylamine | 4-dimethylaminocinnamaldehyde | 0.684 | 48.8% |
| A21 | 4-aminodiphenylamine | Terephthalaldehyde | 0.680 | 47.6% |
| B | 5-aminoindole | Indole-3-carboxaldehyde | 0.675 | −3.6% |
| D6 | 4-aminodiphenylamine | 2-methoxycinnamaldehyde | 0.668 | 41.8% |
| C | 6-aminoindole | Vanillin | 0.667 | −8.0% |
| A | 4-aminoindole | 4-dimethylaminocinnamaldehyde | 0.662 | 39.2% |
| D | 4-aminodiphenylamine | 4-hydroxy-3-methoxycinnamaldehyde | 0.661 | 38.6% |
| A21 | 4-aminoindole | Terephthalaldehyde | 0.651 | 34.3% |
| C | 6-aminoindole | 4-dimethylaminocinnamaldehyde | 0.644 | 31.1% |
| B | 5-aminoindole | 4-dimethylaminocinnamaldehyde | 0.641 | 29.7% |

Concentration dependance of antiozonant activity. The antiozonant activity of Schiff base (SB) of 6-aminoindole and citral was evaluated at two concentrations using solution viscometry. The Schiff base (C3) was prepared as described and was dissolved in 20.0 g/L solutions of Guayule natural rubber in tetrahydrofuran (THF) to achieve an antiozonant concentration of 10 parts per hundred rubber (phr) (1×) or 20 parts per hundred rubber (2×). The initial viscosity of a 50 mL aliquot of this solution was evaluated using a Brookfield LVDVE Viscometer (United States) at 100 rpm. Ozone (O₃) was generated using a 2G Lab Benchtop Ozone Generator (A2Z Ozone, United States) and bubbled through the unstabilized (Blank) and stabilized rubber solutions of 6PPD, and SB at 1 or 2 equimolar concentrations (1×, 2×). The viscosity of the solution was determined after each 60s exposure to O₃ to track GNR degradation throughout the 6-minute experiment. The SB at 2× concentration protected the rubber in solution at equal to better levels than 6PPD.

| | | | Viscometry | Purity |
|---|---|---|---|---|
| | | | φₜ^{α} vs 6PPD | by |
| | Amine & | | @ t = 120, or | LC- |
| No. | aldehyde | structure | as indicated | MS |
| B6 | 5-aminoindole (5AI) & 2-methoxy cinnamaldehyde | 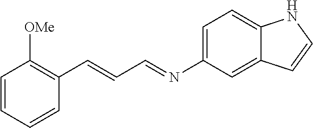 [SMILES: COc1ccccc1/C=C/C=N/c1ccc2[nH]ccc2c1] | 87.3 | 98% |
| C3 | 6-aminoindole (6AI) & Citral | 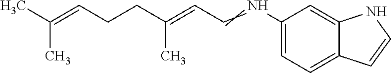 [SMILES: CC(C)=CCC/C(C)=C/C=Nc1ccc2cc[nH]c2c1] | 56.6 | |
| B21 | 5-aminoindole (2) & Terephthalaldehyde | 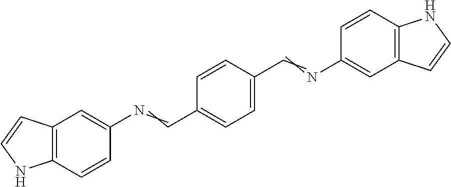 [SMILES: C(=Nc1ccc2[nH]ccc2c1)c1ccc(C=Nc2ccc3[nH]ccc3c2)cc1] | 73.7 | 73% |
| B16 | 5-aminoindole & 1-methyl-1H- benzimidazole-2- carbaldehyde | 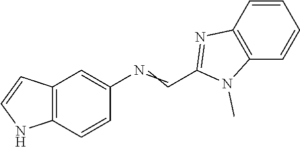 [SMILES: Cn1c(C=Nc2ccc3[nH]ccc3c2)nc2ccccc21] | 54.8 | 99% |
| C24 | 6-aminoindole (2) & glyoxal | 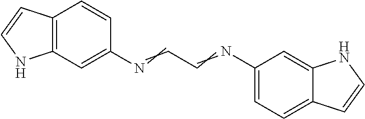 [SMILES: C(C=Nc1ccc2cc[nH]c2c1)=Nc1ccc2cc[nH]c2c1] | 43 | 99% |
| C6 | 6-aminoindole & 2-methoxy- cinnamaldehyde | 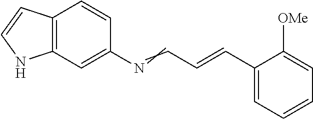 [SMILES: COc1ccccc1/C=C/C=Nc1ccc2cc[nH]c2c1] | 39.4 | 99% |
| C18 | 6AI & Indole-3- carboxaldehyde | 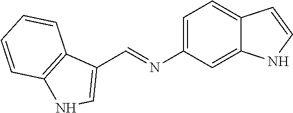 [SMILES: C(=N\c1ccc2cc[nH]c2c1)/c1c[nH]c2ccccc12] | 21.3 | 92% |
| C21 | 6-aminoindole (2x) & terephthalaldehyde | 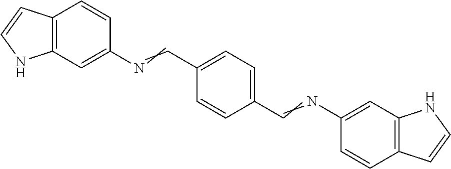 [SMILES: C(=Nc1ccc2cc[nH]c2c1)c1ccc(C=Nc2ccc3cc[nH]c3c2)cc1] | 16.7 | 87% |
| B12 | 5-aminoindole & vanillin | 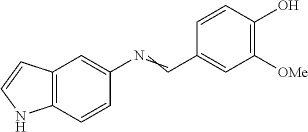 [SMILES: COc1cc(C=Nc2ccc3[nH]ccc3c2)ccc1O] | | 92% |
| D21 | 4-ADPA (2x) & terephthaldehyde | 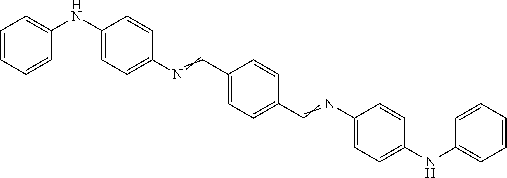 [SMILES: C(=Nc1ccc(Nc2ccccc2)cc1)c1ccc(C=Nc2ccc(Nc3ccccc3)cc2)cc1] | 47.6 | 94% |
| C12 | 6-aminoindole & vanillin | 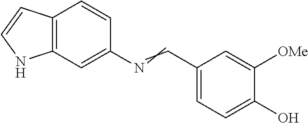 [SMILES: COc1cc(C=Nc2ccc3cc[nH]c3c2)ccc1O] | −8 | 99% |
| A8 | 4-aminoindole (4AI) & 4-dimethyl- aminocinnam- aldehyde | 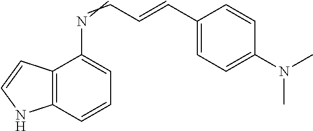 [SMILES: CN(C)c1ccc(/C=C/C=Nc2cccc3[nH]ccc23)cc1] | 39 | 89% |
| A21 | 4-aminoindole (2x) & terephthalaldehyde | 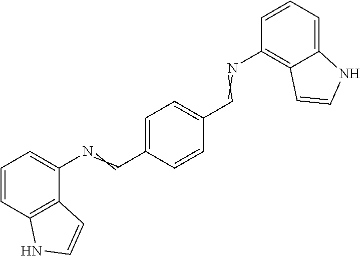 [SMILES: C(=Nc1cccc2[nH]ccc12)c1ccc(C=Nc2cccc3[nH]ccc23)cc1] | 34.3 | 70% |
| C28 | 6AI & Trihydroxy- benzaldehyde | 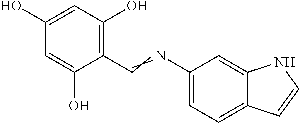 [SMILES: Oc1cc(O)c(C=Nc2ccc3cc[nH]c3c2)c(O)c1] | 98 | |
| C25 | 6AI & Trimethoxy- benzaldehyde | 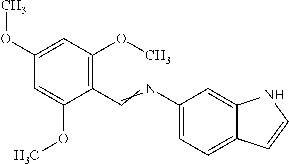 [SMILES: COc1cc(OC)c(C=Nc2ccc3cc[nH]c3c2)c(OC)c1] | 77 | 99.8% |
| B18 | 5AI & Indole-3- carboxaldehyde | 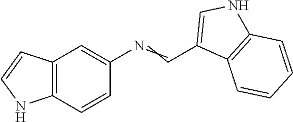 [SMILES: C(=Nc1ccc2[nH]ccc2c1)c1c[nH]c2ccccc12] | −3.6 | 94% |
| C12 | 6AI & vanillin | 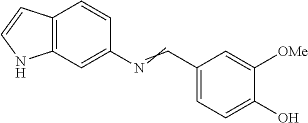 [SMILES: COc1cc(C=Nc2ccc3cc[nH]c3c2)ccc1O] | 91 | 99% |
| C7 | 6AI & 4-hydroxy-3- methoxycinnamaldehyde | 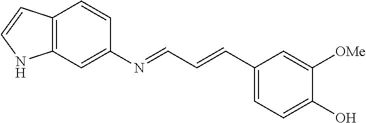 [SMILES: COc1cc(/C=C/C=N/c2ccc3cc[nH]c3c2)ccc1O] | | 99% |
| B7 | 5AI & 4-hydroxy-3- methoxycinnamaldehyde | 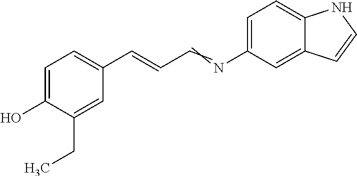 [SMILES: CCc1cc(/C=C/C=Nc2ccc3[nH]ccc3c2)ccc1O] | 5.8 | 93% |
| B32 | 5AI & 3,5-ditBu-4- hydroxybenzaldehyde | 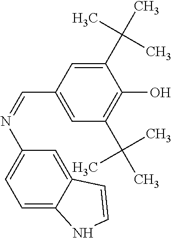 [SMILES: CC(C)(C)c1cc(/C=N\c2ccc3[nH]ccc3c2)cc(C(C)(C)C)c1O] | | 82% |
| B33 | 5AI & alpha-hexyl- cinnamaldehyde | 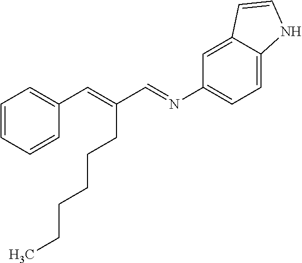 [SMILES: CCCCCCC(/C=N/c1ccc2[nH]ccc2c1)=C\c1ccccc1] | | 95% |
| B34 | 5AI & 2,5- bis(octyloxy) terephthaldehyde | 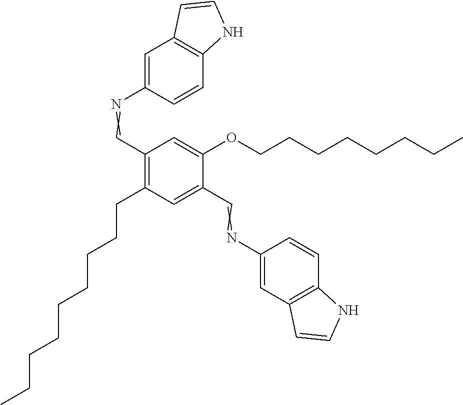 [SMILES: CCCCCCCCCc1cc(C=Nc2ccc3[nH]ccc3c2)c(OCCCCCCCC)cc1C=Nc1ccc2[nH]ccc2c1] | | 82% |
| B6 (R) | 5AI & 2-methoxy- cinnamaldehyde | 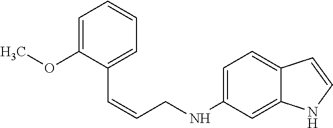 [SMILES: COc1ccccc1/C=C\CNc1ccc2cc[nH]c2c1] | 92.6 | 50% |

Other embodiments are shown in the table below.

| | | | | mp |
|---|---|---|---|---|
| | | | | (C)/ |
| | | | | purity |
| | | | | by |
| | amine | | | LC- |
| No. | aldehyde | structure | Viscometry | MS |
| C21 | 6-aminoindole (2x) & terephthalaldehyde | 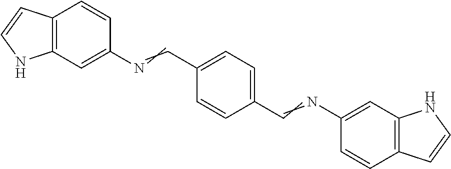 [SMILES: C(=Nc1ccc2cc[nH]c2c1)c1ccc(C=Nc2ccc3cc[nH]c3c2)cc1] | 16.7 | 90% |
| C24 | 6-aminoindole & glyoxal | 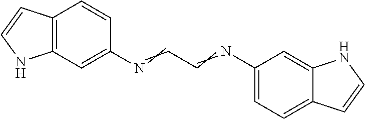 [SMILES: C(C=Nc1ccc2cc[nH]c2c1)=Nc1ccc2cc[nH]c2c1] | 43 | 98.8% |
| B12 | 5-aminoindole & vanillin | 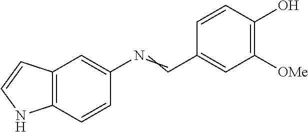 [SMILES: COc1cc(C=Nc2ccc3[nH]ccc3c2)ccc1O] | | 91.6% |
| B18 | 5-aminoindole & Indole-3-carbox- aldehyde | 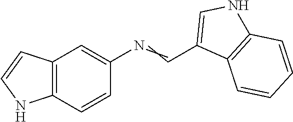 [SMILES: C(=Nc1ccc2[nH]ccc2c1)c1c[nH]c2ccccc12] | −4 | 93.8% |
| C18 | 6-aminoindole & Indole-3-carbox- aldehyde | 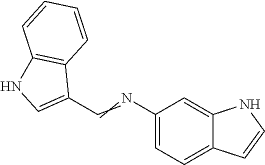 [SMILES: C(=Nc1ccc2cc[nH]c2c1)c1c[nH]c2ccccc12] | 21 | 92.3% |
| D6 | 4-aminodiphenyl- Amine & 2-methoxy- cinnamaldehyde | 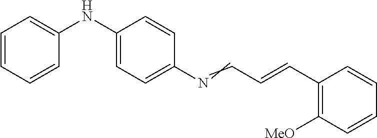 [SMILES: COc1ccccc1/C=C/C=Nc1ccc(Nc2ccccc2)cc1] | 41.8 | 98.6% |
| D3 | 4-aminodiphenyl- amine & citral | 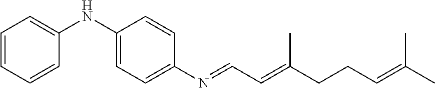 [SMILES: CC(C)=CCC/C(C)=C/C=N/c1ccc(Nc2ccccc2)cc1] | | 73.7% |
| D21 | 4-aminodiphenyl- amine & terepthalaldehyde | 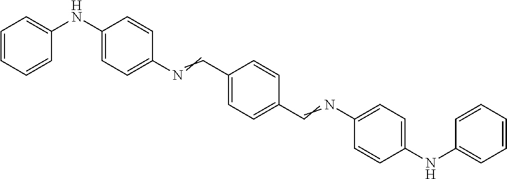 [SMILES: C(=Nc1ccc(Nc2ccccc2)cc1)c1ccc(C=Nc2ccc(Nc3ccccc3)cc2)cc1] | 47.6 | 93.8% |
| D7 | 4-aminodiphenylamine & 4-hydroxy-3-methoxy- cinnamaldehyde | 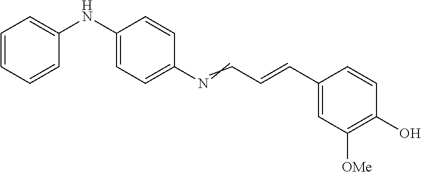 [SMILES: COc1cc(/C=C/C=Nc2ccc(Nc3ccccc3)cc2)ccc1O] | 39 | 98.4% |
| D18 | 4-aminodiphenyl- amine & Indole-3-carboxaldehyde | 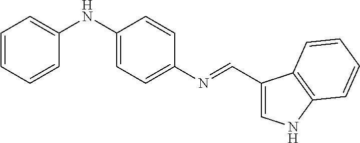 [SMILES: C(=N\c1ccc(Nc2ccccc2)cc1)/c1c[nH]c2ccccc12] | | 97% |
| A8 | 4-aminoindole & 4-dimethylamino- cinnamaldehyde | 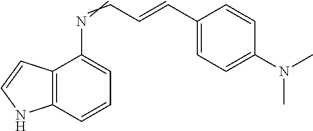 [SMILES: CN(C)c1ccc(/C=C/C=Nc2cccc3[nH]ccc23)cc1] | 39 | 89.4% |
| D8 | 4-aminodiphenylamine & 4-dimethylamino- cinnamaldehyde | 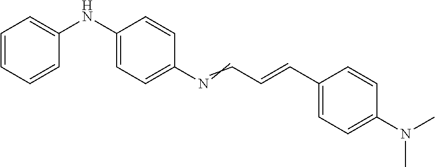 [SMILES: CN(C)c1ccc(/C=C/C=Nc2ccc(Nc3ccccc3)cc2)cc1] | 49 | 95.7% |
| D24 | 4-amino- Diphenylamine & glyoxal | 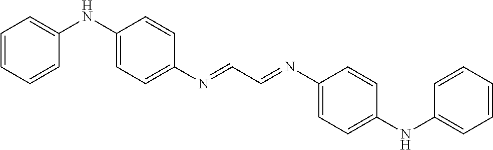 [SMILES: C(/C=N/c1ccc(Nc2ccccc2)cc1)=N\c1ccc(Nc2ccccc2)cc1] | | 97% |
| A21 | 4-aminoindole & terephtalaldehyde | 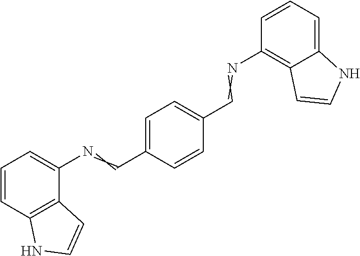 [SMILES: C(=Nc1cccc2[nH]ccc12)c1ccc(C=Nc2cccc3[nH]ccc23)cc1] | 34.3 | 70% |
| B8 | 5-aminoindole & 4-dimethylamino- cinnamaldehyde | 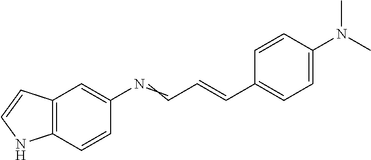 [SMILES: CN(C)c1ccc(/C=C/C=Nc2ccc3[nH]ccc3c2)cc1] | 30 | 98.7% |
| C8 | 6-aminoindole & 4-dimethylamino- cinnamaldehyde | 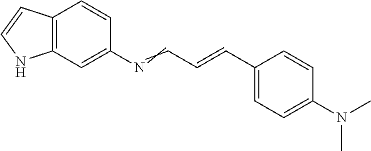 [SMILES: CN(C)c1ccc(/C=C/C=Nc2ccc3cc[nH]c3c2)cc1] | 82 | 97.9% |
| C14 | 6-aminoindole & salicylaldehyde | 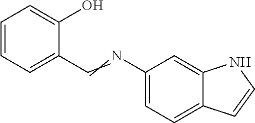 [SMILES: Oc1ccccc1C=Nc1ccc2cc[nH]c2c1] | 79.7 | |
| C11 | 6-aminoindole & cuminaldehyde | 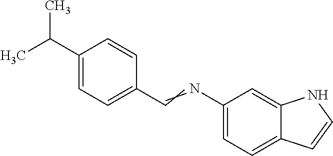 [SMILES: CC(C)c1ccc(C=Nc2ccc3cc[nH]c3c2)cc1] | 40.3 | 98.3% |
| B11 | 5-aminoindole & cuminaldehyde | 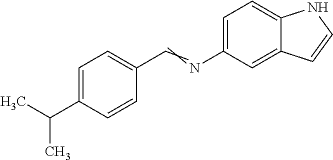 [SMILES: CC(C)c1ccc(C=Nc2ccc3[nH]ccc3c2)cc1] | 80.9 | |
| B31 | 5-aminoindole & 2,4,6-trimethoxy- benzaldehyde | 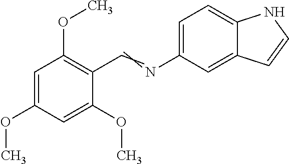 [SMILES: COc1cc(OC)c(C=Nc2ccc3[nH]ccc3c2)c(OC)c1] | 71.3 at t = 200s | |
| C31 | 6-aminoindole & 2,4,6-trimethoxy- benzaldehyde | 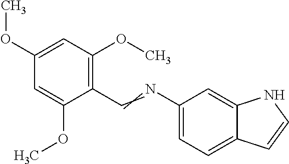 [SMILES: COc1cc(OC)c(C=Nc2ccc3cc[nH]c3c2)c(OC)c1] | 77 | 99.8% |
| B15 | 5-aminoindole & perillaldehyde | 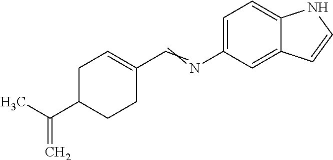 [SMILES: C=C(C)C1CC=C(C=Nc2ccc3[nH]ccc3c2)CC1] | 95.3 | 92.7% |
| C15 | 6-aminoindole & perillaldehyde | 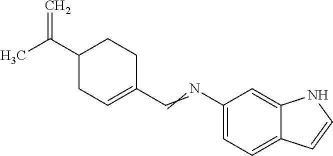 [SMILES: C=C(C)C1CC=C(C=Nc2ccc3cc[nH]c3c2)CC1] | | 83.3% |
| C22 | 6-aminoindole & isophthaldehyde | 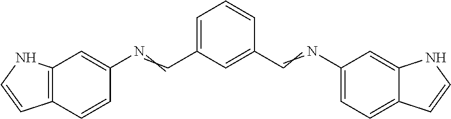 [SMILES: C(=Nc1ccc2cc[nH]c2c1)c1cccc(C=Nc2ccc3cc[nH]c3c2)c1] | 33.4 at t = 200s | 94.3% |
| B19 | 5-aminoindole & Pyrrole-2- carboxaldehyde | 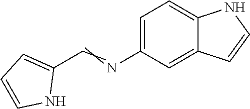 [SMILES: C(=Nc1ccc2[nH]ccc2c1)c1ccc[nH]1] | 67.7 at t = 200s | 98.7% |
| C19 | 6-aminoindole & Pyrrole-2- carboxaldehyde | 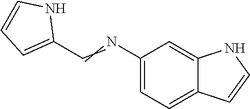 [SMILES: C(=Nc1ccc2cc[nH]c2c1)c1ccc[nH]1] | | 56.4% |
| C20 | 6-aminoindole & Thieno[3.2]thiophene- 2,5-dicarboxaldehyde | 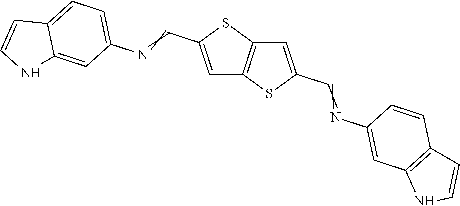 [SMILES: C(=Nc1ccc2cc[nH]c2c1)c1cc2sc(C=Nc3ccc4cc[nH]c4c3)cc2s1] | 65.0 | 97.7% |
| B20 | 5-aminoindole & Thieno[3.2]thiophene- 2,5-dicarboxaldehyde | 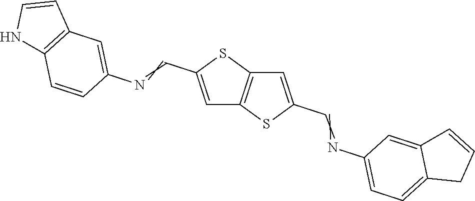 [SMILES: C1=Cc2cc(N=Cc3cc4sc(C=Nc5ccc6[nH]ccc6c5)cc4s3)ccc2C1] | Didn't dissolve completely in solution | 94.0% |
| B17 | 5-aminoindole & Indole-2-carboxaldehyde | 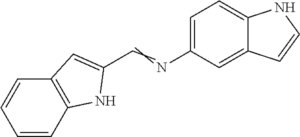 [SMILES: C(=Nc1ccc2[nH]ccc2c1)c1cc2ccccc2[nH]1] | 80.9 | 209.36 |
| B9 | 5-aminoindole & sinapaldehyde | 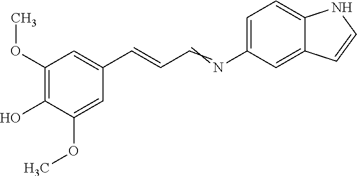 [SMILES: COc1cc(/C=C/C=Nc2ccc3[nH]ccc3c2)cc(OC)c1O] | 81.2 | 189.53 99.0% |
| C9 | 6-aminoindole & sinapaldehyde | 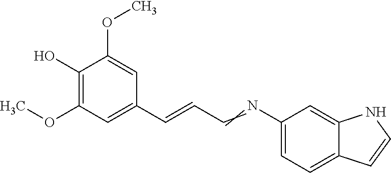 [SMILES: COc1cc(/C=C/C=Nc2ccc3cc[nH]c3c2)cc(OC)c1O] | | 98.0% |
| C2 | 6-aminoindole & 2-methyl-2-pentenal | 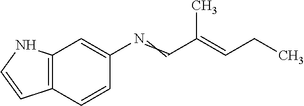 [SMILES: CC/C=C(\C)C=Nc1ccc2cc[nH]c2c1] | | 44.4% |
| F6 | Butylamine & 2-methoxy- cinnamaldehyde | 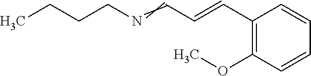 [SMILES: CCCCN=C/C=C/c1ccccc1OC] | | Oily 96.4% |
| E6 | Sec-butylamine & 2-methoxy- cinnamaldehyde | 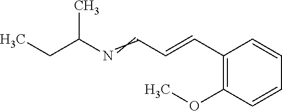 [SMILES: CCC(C)N=C/C=C/c1ccccc1OC] | 75.8 | 95.1% |
| C21 | 6-aminoindole & terephtalaldehyde | 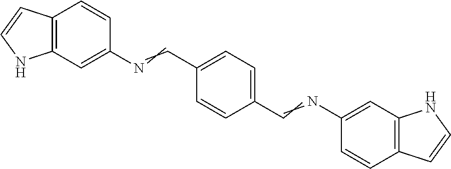 [SMILES: C(=Nc1ccc2cc[nH]c2c1)c1ccc(C=Nc2ccc3cc[nH]c3c2)cc1] | 16.7 | 87% |
| E6 | Sec-butylamine & 2-methoxy- cinnamaldehyde | 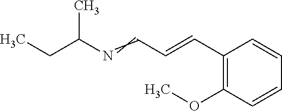 [SMILES: CCC(C)N=C/C=C/c1ccccc1OC] | 75.8 | 97.6% |
| F6 | Butylamine & 2-methoxy- cinnamaldehyde | 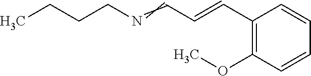 [SMILES: CCCCN=C/C=C/c1ccccc1OC] | | 99.2% |
| G3 | Thiosemicarbazide & citral | 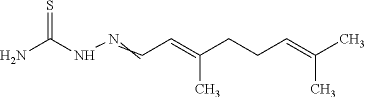 | 80.9 | 99.7% |
| G14 | Thiosemicarbazide & Salicylaldehyde (1x) | 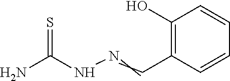 [SMILES: NC(=S)NN=Cc1ccccc1O] | 82.6 | 211 99.5% |
| H6 | 1-(2-aminophenyl)- pyrrole & 2-methoxy- cinnamaldehyde | 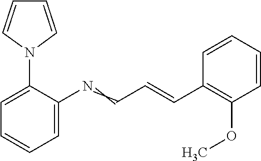 [SMILES: COc1ccccc1/C=C/C=Nc1ccccc1-n1cccc1] | 65.53 @200s | 75% |
| H19 | 1-(2-aminophenyl)- Pyrrole & Pyrrole-2-carboxaldehyde | 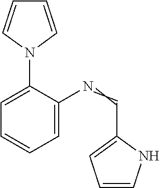 [SMILES: C(=Nc1ccccc1-n1cccc1)c1ccc[nH]1] | 78.0 @200s | |
| H8 | 1-(2-aminophenyl)- pyrrole & 4-dimethyl- aminocinnamaldehyde | 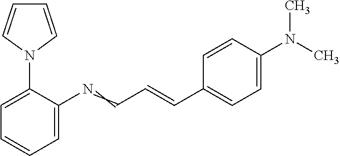 [SMILES: CN(C)c1ccc(/C=C/C=Nc2ccccc2-n2cccc2)cc1] | 74.5 @200s | 77% |
| J6 | 1-aminopyrrole & 2-methoxy- cinnamaldehyde | 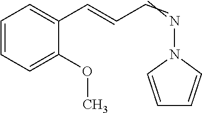 [SMILES: COc1ccccc1/C=C/C=Nn1cccc1] | 70.7 @200s | 98% |
| J8 | 1-aminopyrrole & 4-dimethyl- aminocinnamaldehyde | 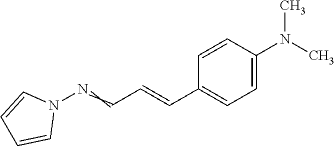 [SMILES: CN(C)c1ccc(/C=C/C=Nn2cccc2)cc1] | 89.0 @200s | 97% |
| B51 | 5-aminoindole & 2-hydroxy-5- methoxybenzaldehyde | 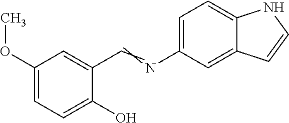 [SMILES: COc1ccc(O)c(C=Nc2ccc3[nH]ccc3c2)c1] | 87.44 @200s | |
| E6 | Sec-butylamine & 2-methoxy- cinnamaldehyde | 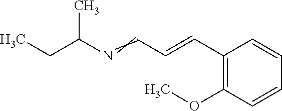 [SMILES: CCC(C)N=C/C=C/c1ccccc1OC] | 75.8 | |
| B6 (2) | 5-aminoindole & 2-methoxy- cinnamaldehyde (2x) | 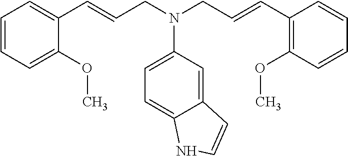 [SMILES: COc1ccccc1/C=C/CN(C/C=C/c1ccccc1OC)c1ccc2[nH]ccc2c1] | | 20% |
| B37 | 5-aminoindole & Alpha-methyl cinnamaldehyde | 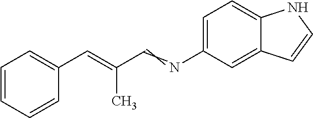 [SMILES: C/C(C=Nc1ccc2[nH]ccc2c1)=C\c1ccccc1] | 71.4 | 98.3% |
| B38 | 5-aminoindole & 4-fluoro- cinnamaldehyde | 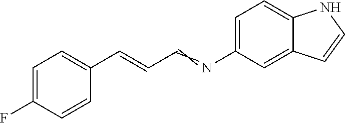 [SMILES: Fc1ccc(/C=C/C=Nc2ccc3[nH]ccc3c2)cc1] | 71.3 | 98.6% |
| B39 | 5-aminoindole & Trans-p-methoxy- cinnamaldehyde | 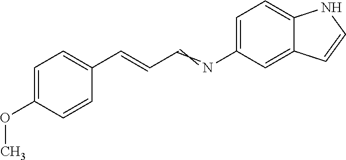 [SMILES: COc1ccc(/C=C/C=Nc2ccc3[nH]ccc3c2)cc1] | 74.6 | 98.8% |
| B40 | 5-aminoindole & Alpha-hexyl cinnamaldehyde | 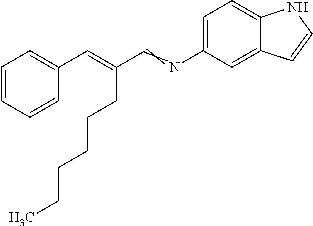 [SMILES: CCCCCC/C(C=Nc1ccc2[nH]ccc2c1)=C\c1ccccc1] | 72.1 | 99.5% |
| E37 | Sec-butylamine &Alpha-methyl cinnamaldehyde | 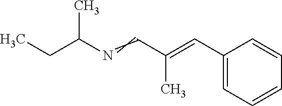 | 89.37 | |
| E38 | Sec-butylamine & 4-fluorocinnamaldehyde | 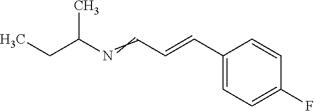 | 81.63 | |
| E33 | Sec-butylamine Alpha- hexylcinnamaldehyde | 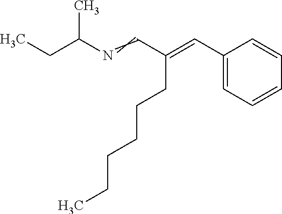 [SMILES: CCCCCC/C(C=NC(C)CC)=C\c1ccccc1] | 81.63 | |
| B41 | 5-aminoindole & 4-acetamido- benzaldehyde | 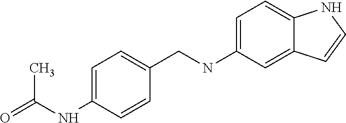 [SMILES: CC(=O)Nc1ccc(CNc2ccc3[nH]ccc3c2)cc1] | 82.3 | |
| B45 | 5-aminoindole & m-tolualdehyde | 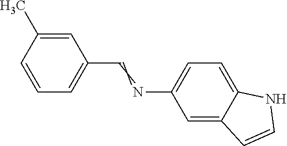 [SMILES: Cc1cccc(C=Nc2ccc3[nH]ccc3c2)c1] | | 93% |
| B42 | 5-aminoindole & Pentamethyl- benzaldehyde | 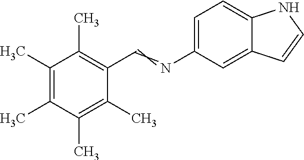 [SMILES: Cc1c(C)c(C)c(C=Nc2ccc3[nH]ccc3c2)c(C)c1C] | 82 | 97% |
| B44 | 5-aminoindole & 3,4-dibenzyloxy- benzaldehyde | 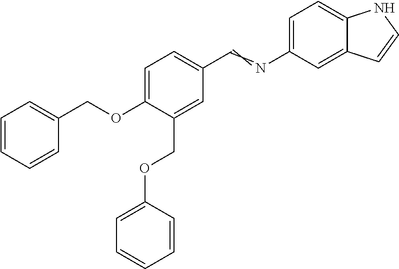 [SMILES: C(=Nc1ccc2[nH]ccc2c1)c1ccc(OCc2ccccc2)c(COc2ccccc2)c1] | | 96% |
| B43 | 5-aminoindole & 4-(dimethylamino)- benzaldehyde | 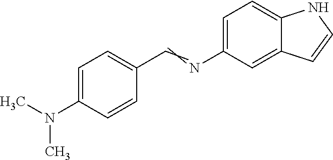 [SMILES: CN(C)c1ccc(C=Nc2ccc3[nH]ccc3c2)cc1] | 85.6 | |
| | | | | Purity |
| Our | Amine & | | | by LC- |
| code | aldehyde | structure | Viscometry | MS |
| C31R | 6-aminoindole & 2,4,6-trimethoxy- benzaldehyde | 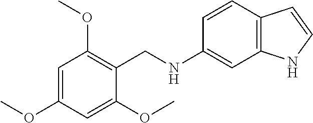 [SMILES: COc1cc(OC)c(CNc2ccc3cc[nH]c3c2)c(OC)c1] | 99.9 | 97% |
| C22R | 6-aminoindole & isophthaldehyde | 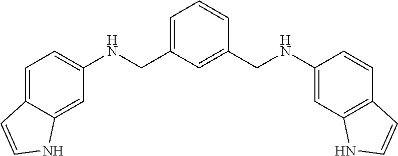 [SMILES: c1cc(CNc2ccc3cc[nH]c3c2)cc(CNc2ccc3cc[nH]c3c2)c1] | 103 | 221 |
| H8R | 1-(2-aminophenyl)- pyrrole & 4-dimethyamino- cinnamaldehyde | 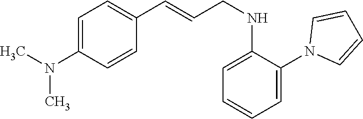 [SMILES: CN(C)c1ccc(/C=C/CNc2ccccc2-n2cccc2)cc1] | 57.5 at t = 200s | |
| J6R | 1-aminopyrrole & 2-methoxy- cinnamaldehyde | 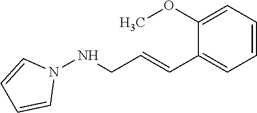 [SMILES: COc1ccccc1/C=C/CNn1cccc1] | 71.5 @ 200s | |
| E6R | Sec-butylamine & 2-methoxy- cinnamaldehyde | 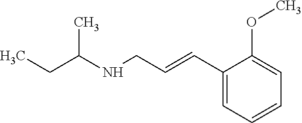 [SMILES: CCC(C)NC/C=C/c1ccccc1OC] | 87 | |
| C22R | 6-aminoindole & Isophthalaldehyde | 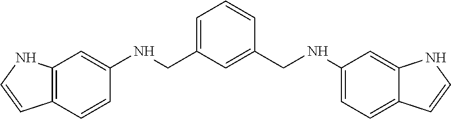 [SMILES: c1cc(CNc2ccc3cc[nH]c3c2)cc(CNc2ccc3cc[nH]c3c2)c1] | 103 | 221 85% |
| B21R | 5-aminoindole & Terephthalaldehyde | 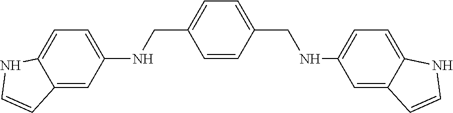 [SMILES: c1cc2cc(NCc3ccc(CNc4ccc5[nH]ccc5c4)cc3)ccc2[nH]1] | | 75% |
| B37R | 5-aminoindole & alpha-methyl- cinnamaldehyde | 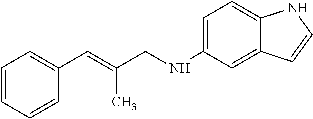 [SMILES: C/C(=C\c1ccccc1)CNc1ccc2[nH]ccc2c1] | 94.1 | 97.4% |
| B38R | 5-aminoindole & 4-fluoro- cinnamaldehyde | 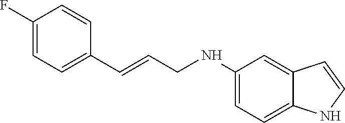 [SMILES: Fc1ccc(/C=C/CNc2ccc3[nH]ccc3c2)cc1] | 94.0 | 98.5% |
| B39R | 5-aminoindole & trans-p-methoxy- cinnamaldehyde | 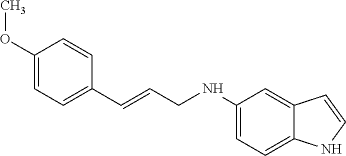 [SMILES: COc1ccc(/C=C/CNc2ccc3[nH]ccc3c2)cc1] | 94.2 | 95.6% |
| B33R | 5-aminoindole & alpha-hexyl- cinnamaldehyde | 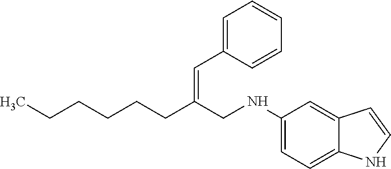 [SMILES: CCCCCC/C(=C/c1ccccc1)CNc1ccc2[nH]ccc2c1] | 95.9 | 93.4% |
| B34R | 5-aminoindole & 2,5-Bis(octyloxy)- terephthalaldehyde | 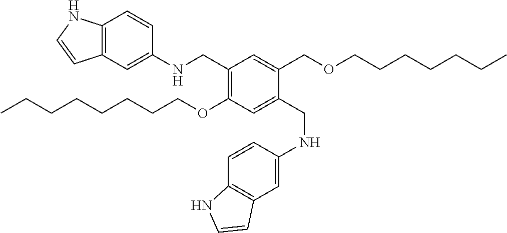 [SMILES: CCCCCCCCOc1cc(CNc2ccc3[nH]ccc3c2)c(COCCCCCCC)cc1CNc1ccc2[nH]ccc2c1] | 84.6 | 66% |
| B41R | 5-aminoindole & 4-acetamido- benzaldehyde | 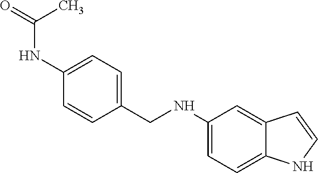 [SMILES: CC(=O)Nc1ccc(CNc2ccc3[nH]ccc3c2)cc1] | 97.1 | |
| B43R | 5-aminoindole & 4-(dimethylamino) benzaldehyde | 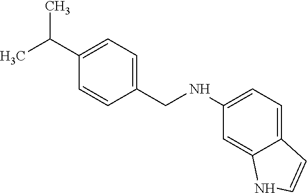 [SMILES: CC(C)c1ccc(CNc2ccc3cc[nH]c3c2)cc1] | 97.1 | |
| B18R | 5-aminoindole & Indole-3- carboxaldehyde | 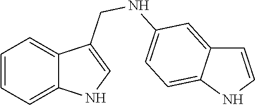 [SMILES: c1ccc2c(CNc3ccc4[nH]ccc4c3)c[nH]c2c1] | 92.2 | |

We claim:

1. A method of treating a rubber polymer to protect it against damage by ozone comprising, treating the polymer with an effective amount of a composition comprising a compound of formula: 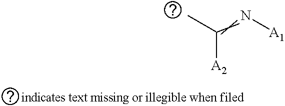
wherein A₁ is 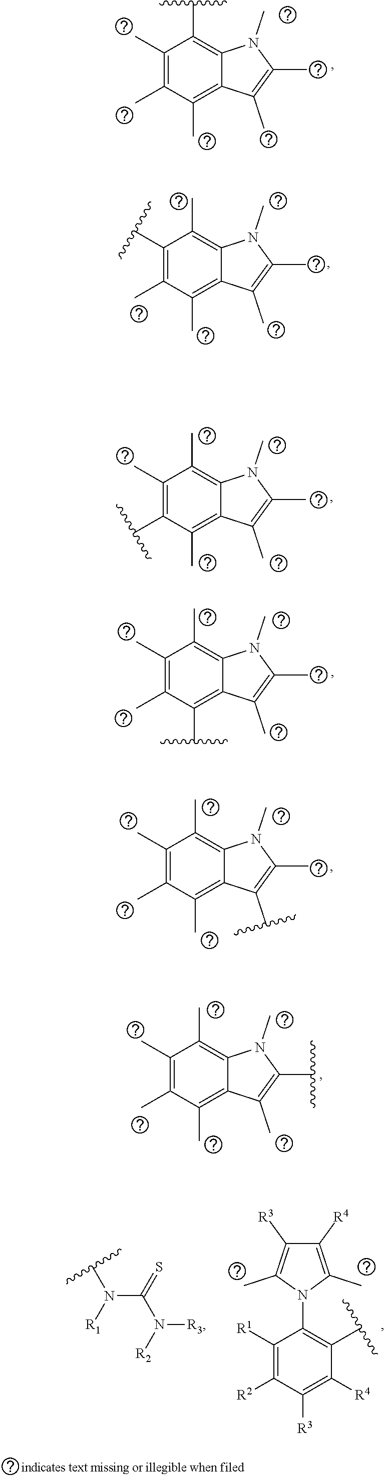
n-butyl, sec-butyl, iso-butyl, tert-butyl groups or a monocyclic heteroaryl group each of which is optionally substituted by one or more of R₁, R₂, R₃, R₄, R₅, R₆,
wherein, A2 and A3 are independently selected from
a) hydrogen;
b) a saturated or unsaturated aliphatic group optionally substituted with one or more unsubstituted or substituted alkyl groups or aryl groups or an imine functional group bearing an A1, A2 or A3 substituent;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, NR₇R₈, acyl, alkyl ether or aryl ether substituents;
wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group;
and wherein R₇, R₈ are H or alkyl.

2. The method of claim 1 wherein the composition comprises compounds with cis or trans isomers of a CN imine bond or a mixture of the isomers.

3. The method of claim 1 with a compound of formula

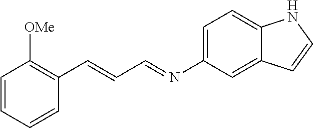 [SMILES: COc1ccccc1/C=C/C=N/c1ccc2[nH]ccc2c1]

4. The method of claim 1 with a compound of formula

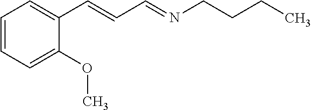 [SMILES: CCCC/N=C/C=C/c1ccccc1OC]

5. The method of claim 1 with a compound of formula

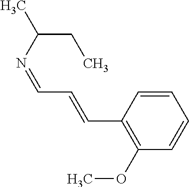 [SMILES: CCC(C)/N=C\C=C\c1ccccc1OC]

6. The method of claim 1 with a compound of formula

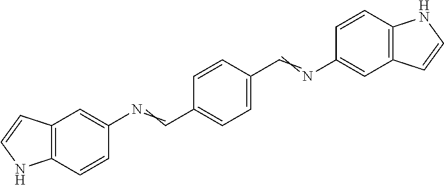 [SMILES: C(=Nc1ccc2[nH]ccc2c1)c1ccc(C=Nc2ccc3[nH]ccc3c2)cc1]

7. The method of claim 1 with a compound of formula

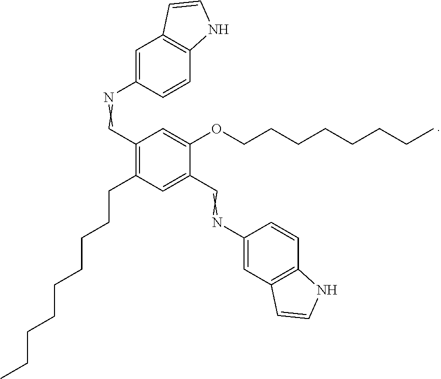 [SMILES: CCCCCCCCCc1cc(C=Nc2ccc3[nH]ccc3c2)c(OCCCCCCCC)cc1C=Nc1ccc2[nH]ccc2c1]

8. A method of treating a rubber polymer to protect it from degradation by ozone by treating it with an effective amount of at least one compound selected from the group comprising of: 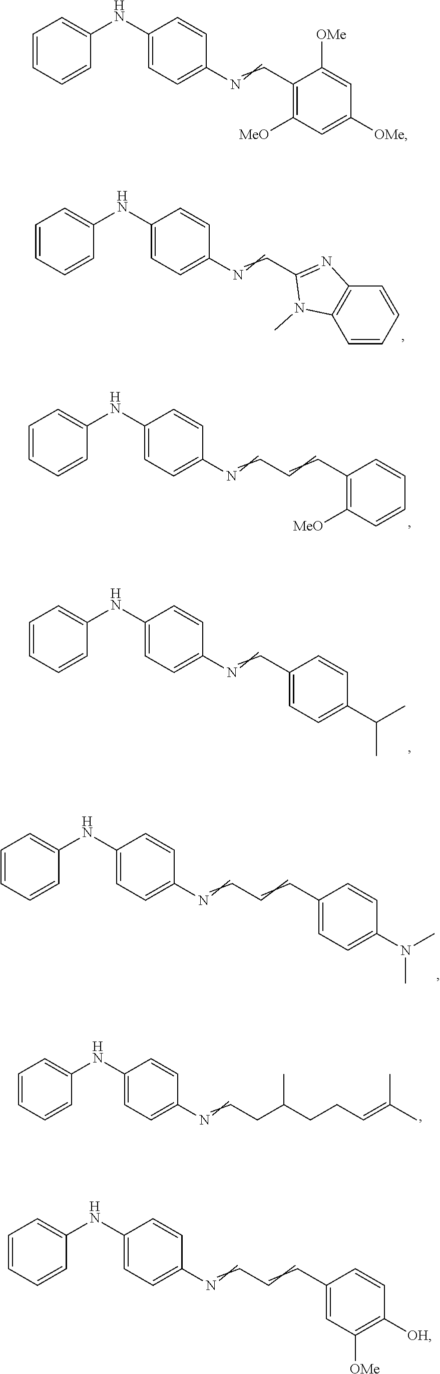 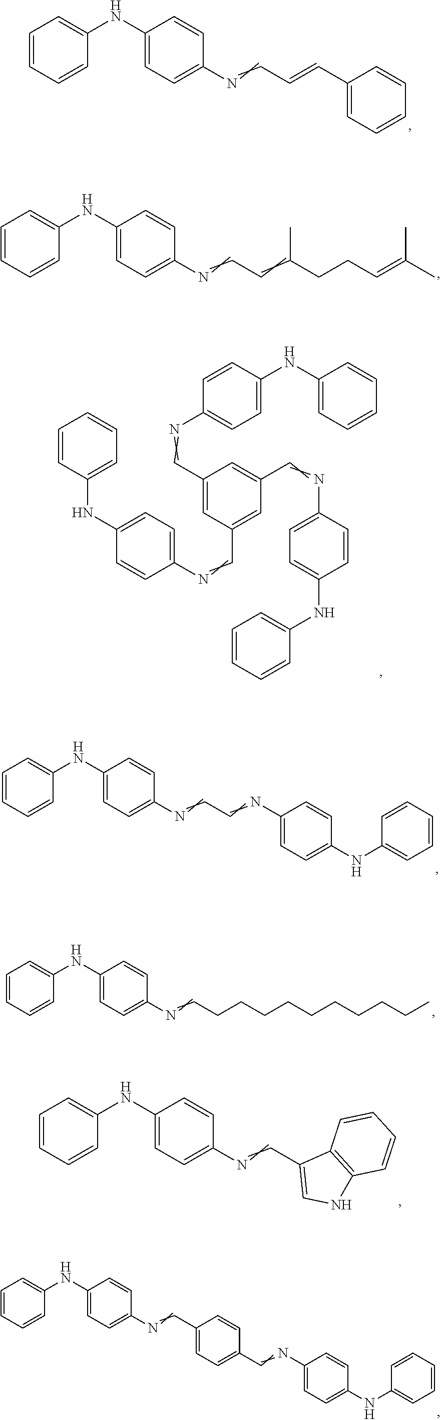 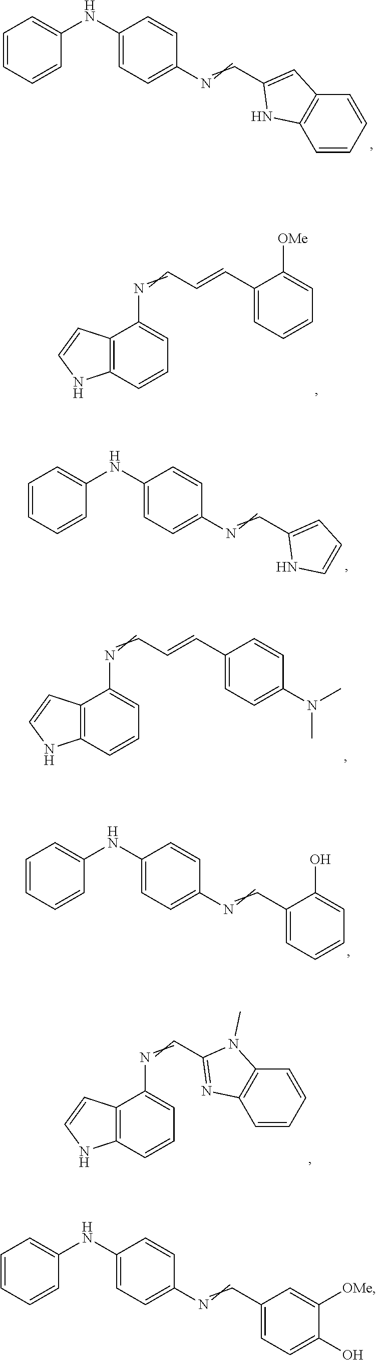 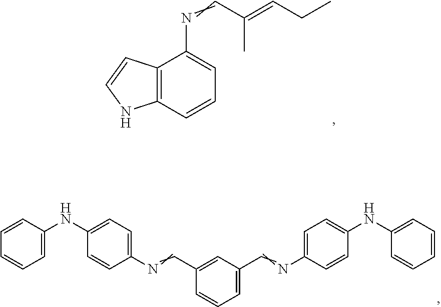 [SMILES: CC/C=C(\C)C=Nc1cccc2[nH]ccc12 ; C(=Nc1ccc(Nc2ccccc2)cc1)c1cccc(C=Nc2ccc(Nc3ccccc3)cc2)c1] 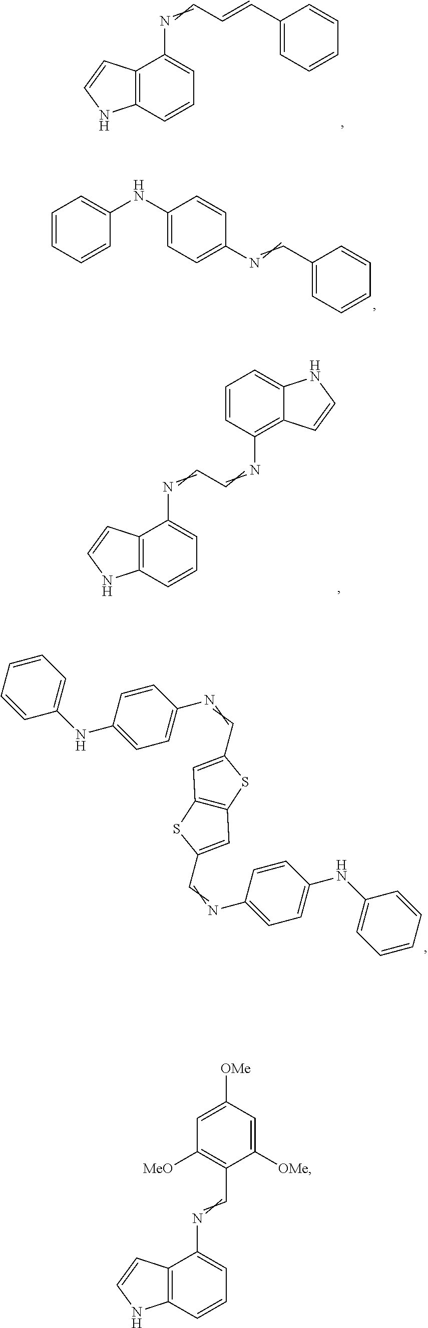 [SMILES: C(/C=C/c1ccccc1)=Nc1cccc2[nH]ccc12 ; C(=Nc1ccc(Nc2ccccc2)cc1)c1ccccc1 ; C(C=Nc1cccc2[nH]ccc12)=Nc1cccc2[nH]ccc12 ; C(=Nc1ccc(Nc2ccccc2)cc1)c1cc2sc(C=Nc3ccc(Nc4ccccc4)cc3)cc2s1 ; COc1cc(OC)c(C=Nc2cccc3[nH]ccc23)c(OC)c1] 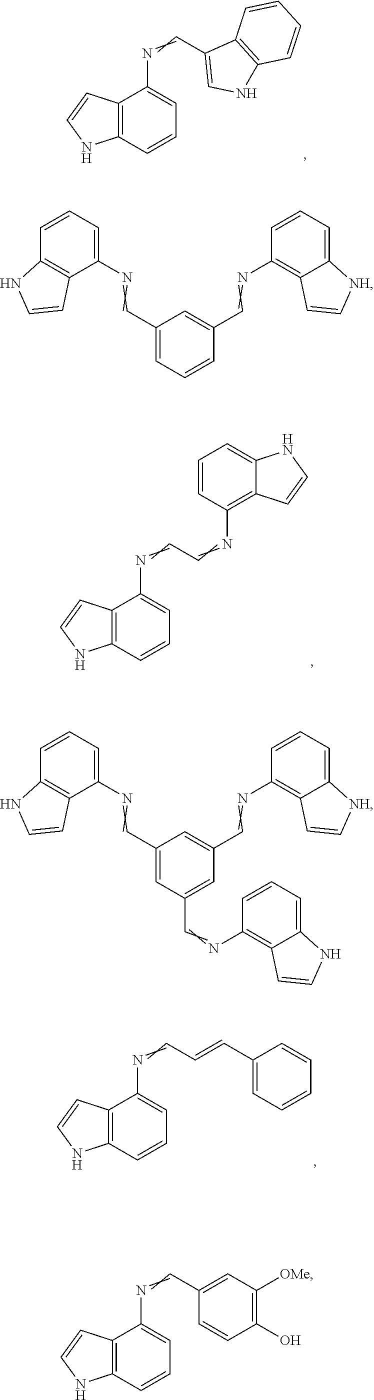 [SMILES: C(=Nc1cccc2[nH]ccc12)c1c[nH]c2ccccc12 ; C(=Nc1cccc2[nH]ccc12)c1cccc(C=Nc2cccc3[nH]ccc23)c1 ; C(C=Nc1cccc2[nH]ccc12)=Nc1cccc2[nH]ccc12 ; C(=Nc1cccc2[nH]ccc12)c1cc(C=Nc2cccc3[nH]ccc23)cc(C=Nc2cccc3[nH]ccc23)c1 ; C(/C=C/c1ccccc1)=Nc1cccc2[nH]ccc12 ; COc1cc(C=Nc2cccc3[nH]ccc23)ccc1O] 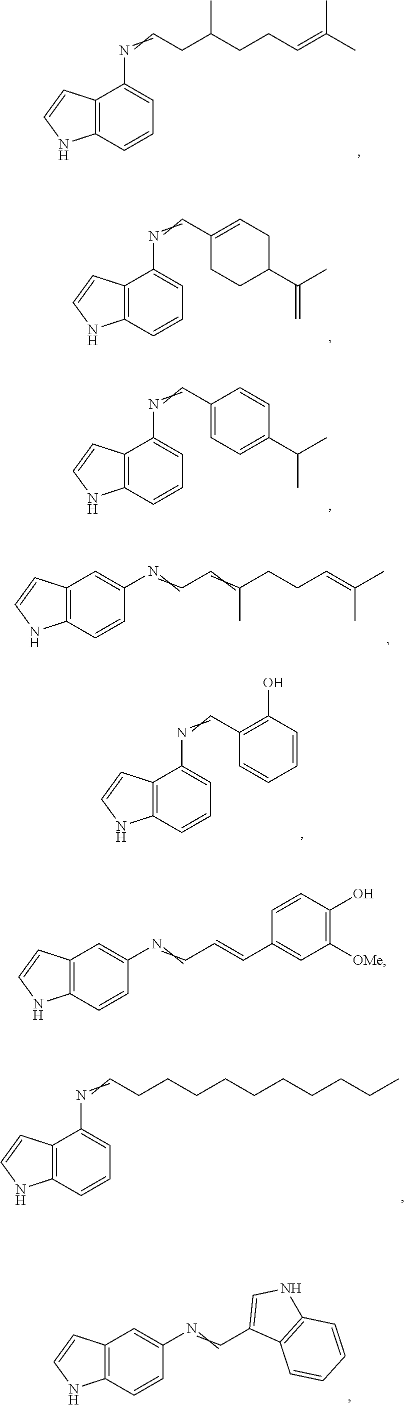 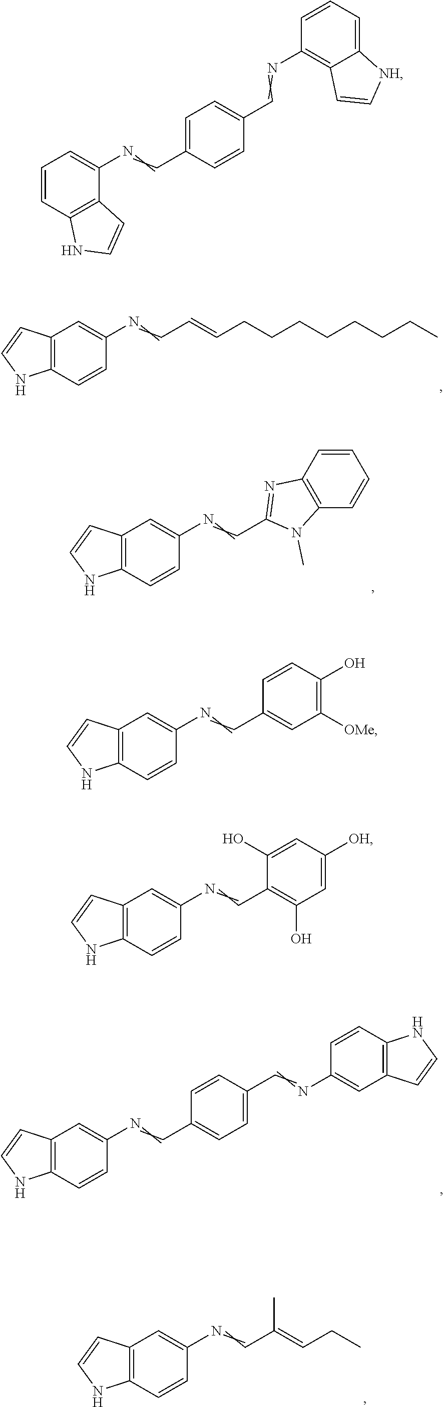 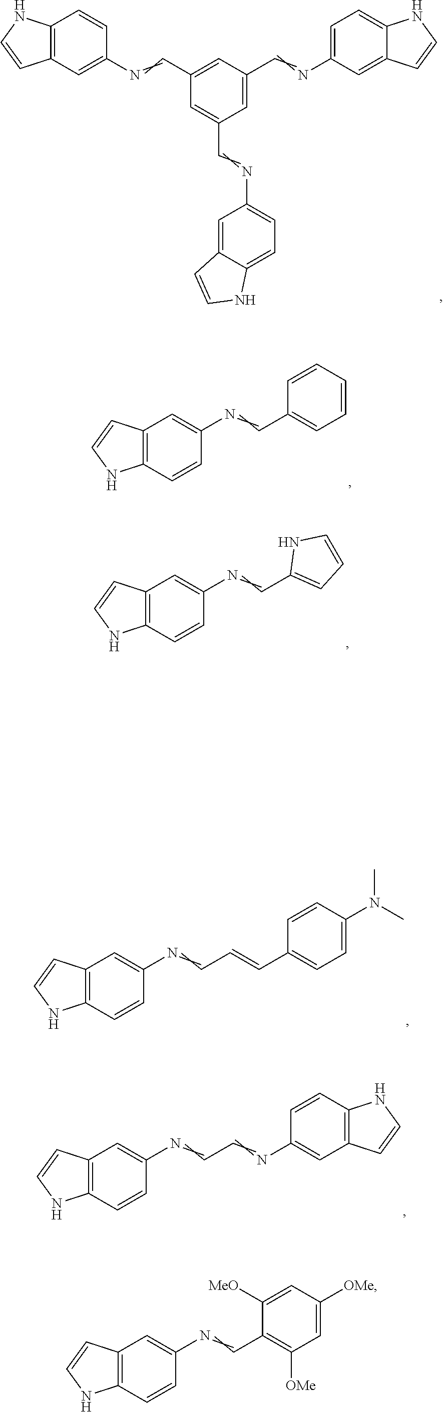 [SMILES: C(=Nc1ccc2[nH]ccc2c1)c1cc(C=Nc2ccc3[nH]ccc3c2)cc(C=Nc2ccc3[nH]ccc3c2)c1 ; C(=Nc1ccc2[nH]ccc2c1)c1ccccc1 ; C(=Nc1ccc2[nH]ccc2c1)c1ccc[nH]1 ; CN(C)c1ccc(/C=C/C=Nc2ccc3[nH]ccc3c2)cc1 ; C(C=Nc1ccc2[nH]ccc2c1)=Nc1ccc2[nH]ccc2c1 ; COc1cc(OC)c(C=Nc2ccc3[nH]ccc3c2)c(OC)c1] 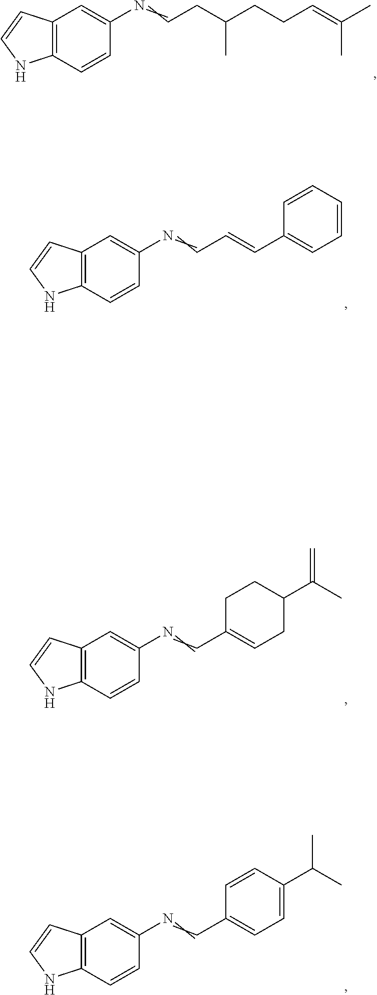 [SMILES: CC(C)=CCCC(C)CC=Nc1ccc2[nH]ccc2c1 ; C(/C=C/c1ccccc1)=Nc1ccc2[nH]ccc2c1 ; C=C(C)C1CC=C(C=Nc2ccc3[nH]ccc3c2)CC1 ; CC(C)c1ccc(C=Nc2ccc3[nH]ccc3c2)cc1] 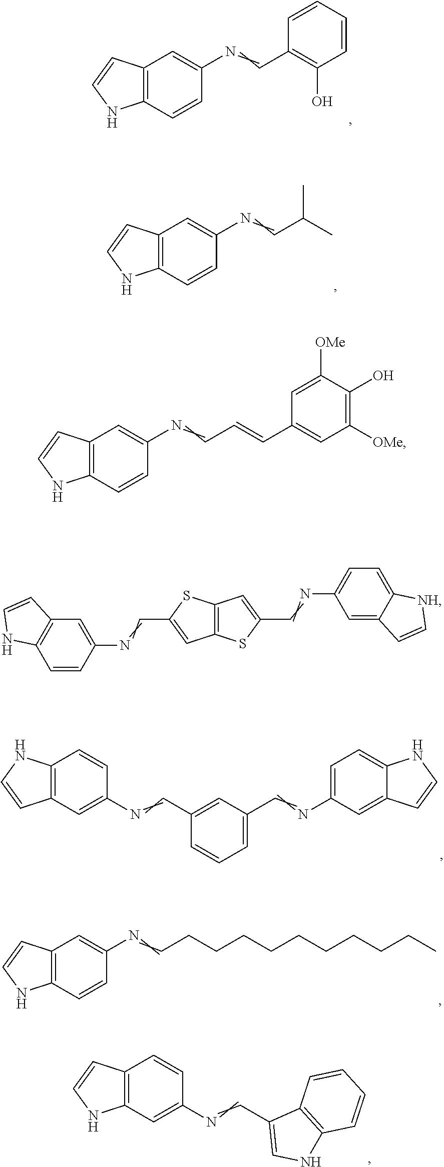 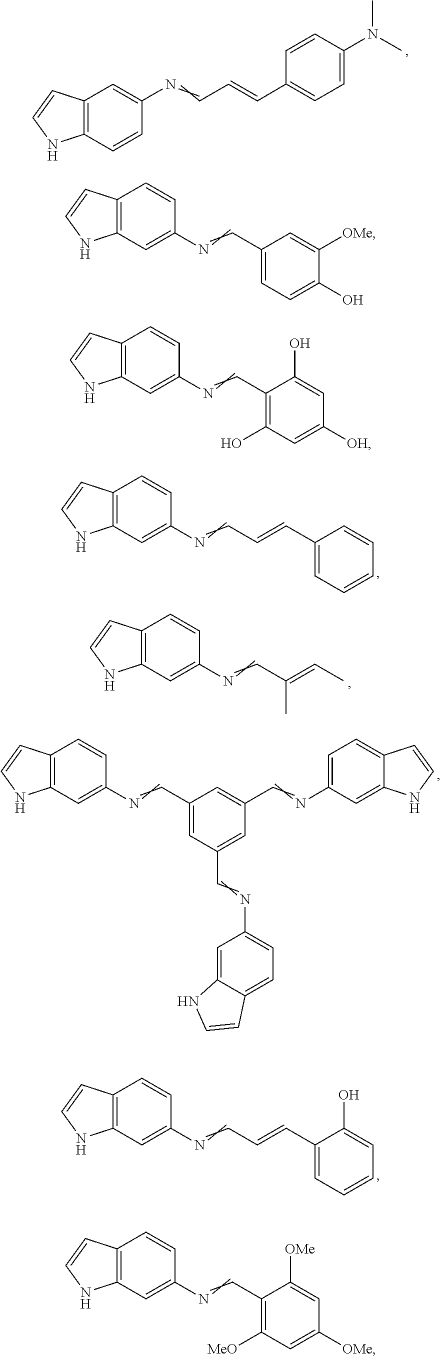 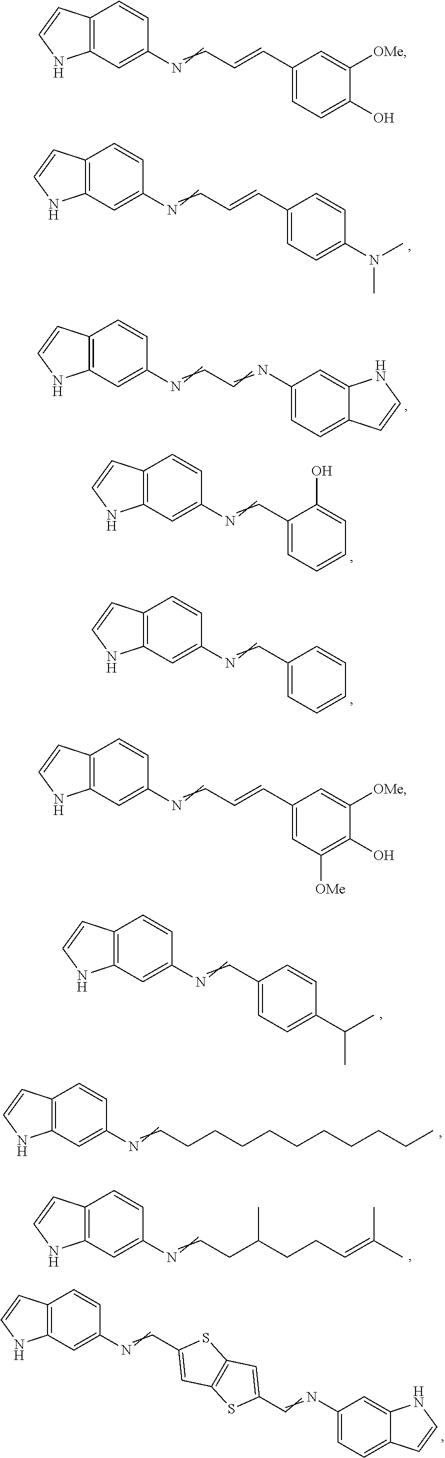 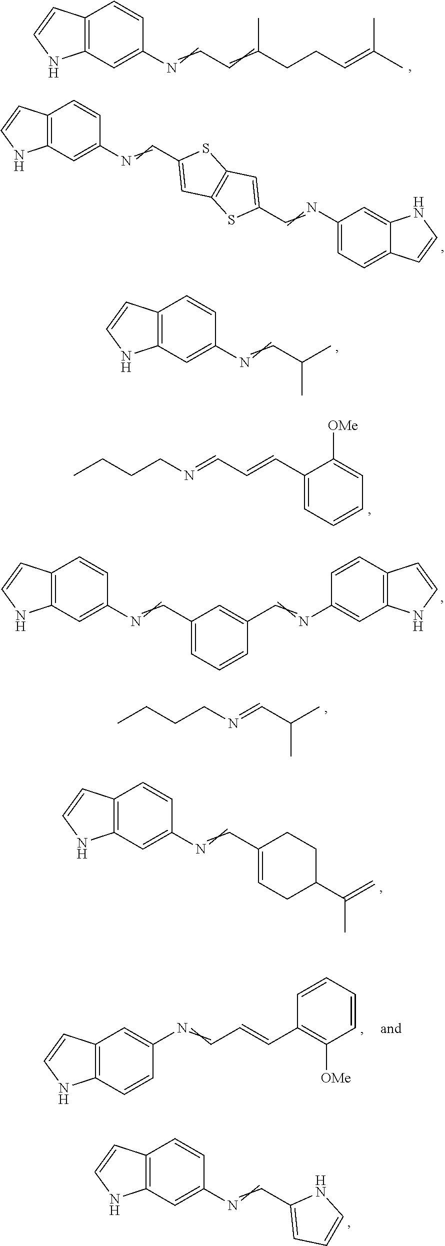
or mixtures thereof wherein the composition comprises cis or trans isomers of CN imine bond(s) or a mixture of the isomers.

9. A rubber polymer formulation comprising an antiozonant compound of formula: 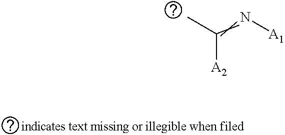
Wherein A₁ is 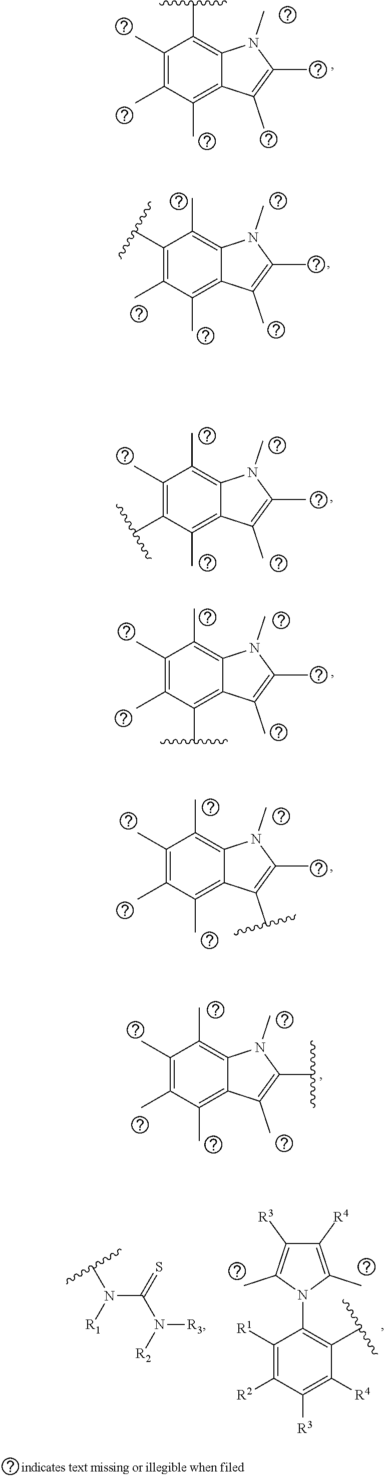
n-butyl, sec-butyl, iso-butyl, tert-butyl groups or a monocyclic heteroaryl group each of which is optionally substituted by one or more of R₁, R₂, R₃, R₄, R₅, R₆,
wherein, A2 and A3 are independently selected from a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
and wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl.

10. The rubber polymer formulation of claim 9 wherein the rubber is a natural rubber.

11. The rubber polymer formulation of claim 9 wherein the polymer is selected from at least one of polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber or mixtures thereof.

12. The rubber polymer formulation of claim 9 as a vulcanizable formulation.

13. The rubber polymer formulation of claim 9 wherein the compound is present in an amount from 0.1 to 10 parts per 100 parts by weight of rubber.

14. The rubber polymer formulation of claim 9 wherein a mixture of 1 or more antiozonant compounds is present in a total amount from 0.1 to 10 parts per 100 parts by weight of rubber.

15. The rubber polymer formulation of claim 9 wherein a mixture of 1 or more antiozonant compounds is present is the total amount of 0.1 to 5 parts per 100 parts by weight of rubber.

16. The rubber polymer formulation of claim 9 in a vulcanized elastomeric article.

17. The rubber polymer formulation of claim 9 in a vehicle tire.

18. The article of claim 17 dyed by applying an effective amount of a formulation of claim.

19. A composition comprising a compound of formula: 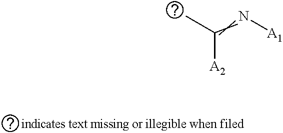
wherein A1 is 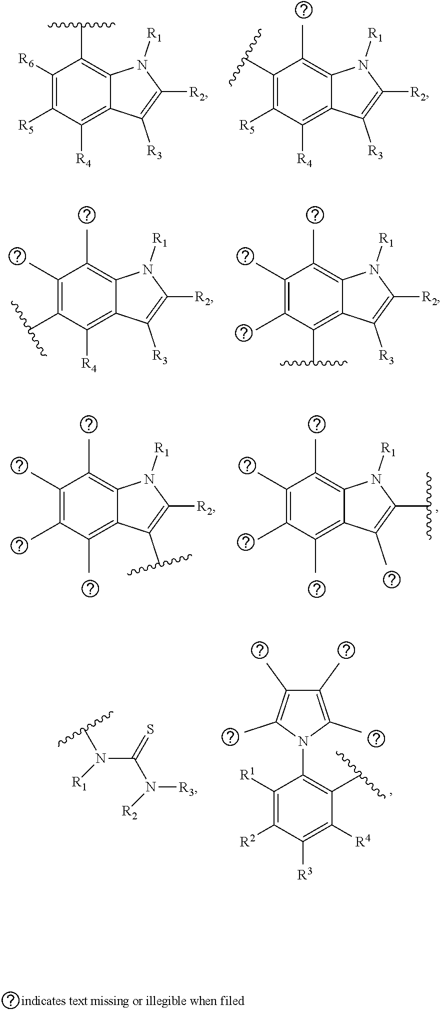
n-butyl, sec-butyl, iso-butyl, tert-butyl groups or a monocyclic heteroaryl group optionally substituted by one or more of R₁, R₂, R₃, R₄, R₅, R₆,
wherein, A2 and A3 are independently selected from a) hydrogen;
b) a saturated or unsaturated aliphatic group optionally substituted with one or more unsubstituted or substituted alkyl groups or aryl groups or an imine functional group bearing A1, A2 or A3 substituents;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
wherein R₁, R₂, R₃, R₄, R₅, R₆ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl.

20. The composition of claim 19 wherein the composition comprises compounds with cis or trans isomers of a CN imine bond or a mixture of the isomers.

21. The composition of claim 19with a compound of formula

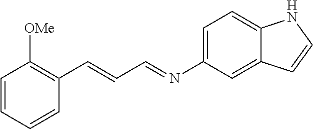 [SMILES: COc1ccccc1/C=C/C=N/c1ccc2[nH]ccc2c1]

22. The method of claim 19 with a compound of formula

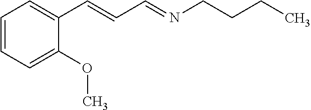 [SMILES: CCCC/N=C/C=C/c1ccccc1OC]

23. The method of claim 19 with a compound of formula

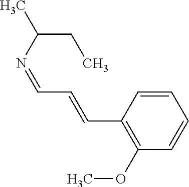 [SMILES: CCC(C)/N=C\C=C\c1ccccc1OC]

24. The method of claim 19 with a compound of formula

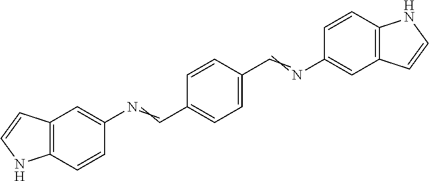 [SMILES: C(=Nc1ccc2[nH]ccc2c1)c1ccc(C=Nc2ccc3[nH]ccc3c2)cc1]

25. The method of claim 19 with a compound of formula

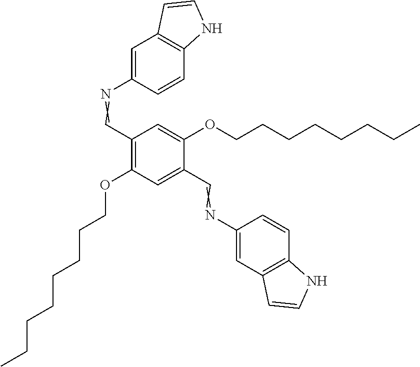 [SMILES: CCCCCCCCOc1cc(C=Nc2ccc3[nH]ccc3c2)c(OCCCCCCCC)cc1C=Nc1ccc2[nH]ccc2c1]

26. The composition of claim 19 with at least one compound selected from the group consisting of:

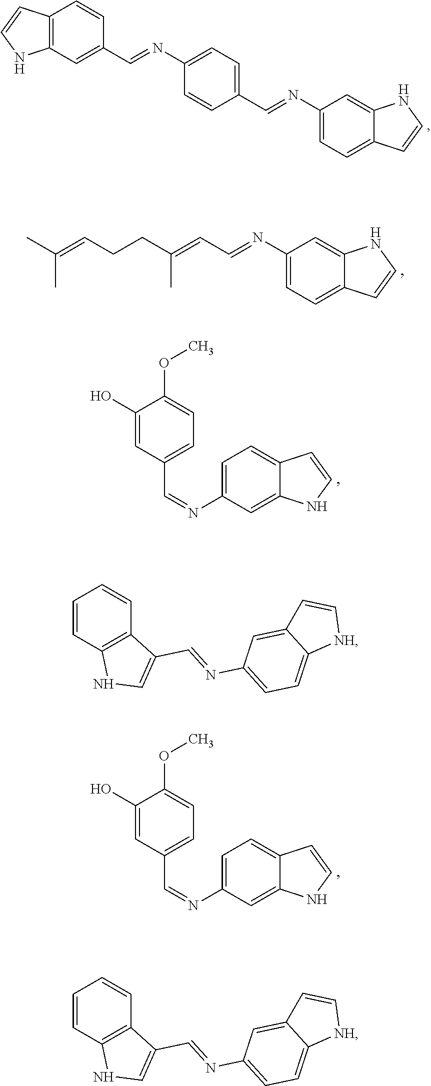 [SMILES: C(=N\c1ccc(/C=N/c2ccc3cc[nH]c3c2)cc1)/c1ccc2cc[nH]c2c1 ; CC(C)=CCC/C(C)=C/C=N/c1ccc2cc[nH]c2c1 ; COc1ccc(/C=N\c2ccc3cc[nH]c3c2)cc1O ; C(=N\c1ccc2[nH]ccc2c1)/c1c[nH]c2ccccc12 ; COc1ccc(/C=N\c2ccc3cc[nH]c3c2)cc1O ; C(=N\c1ccc2[nH]ccc2c1)/c1c[nH]c2ccccc12]

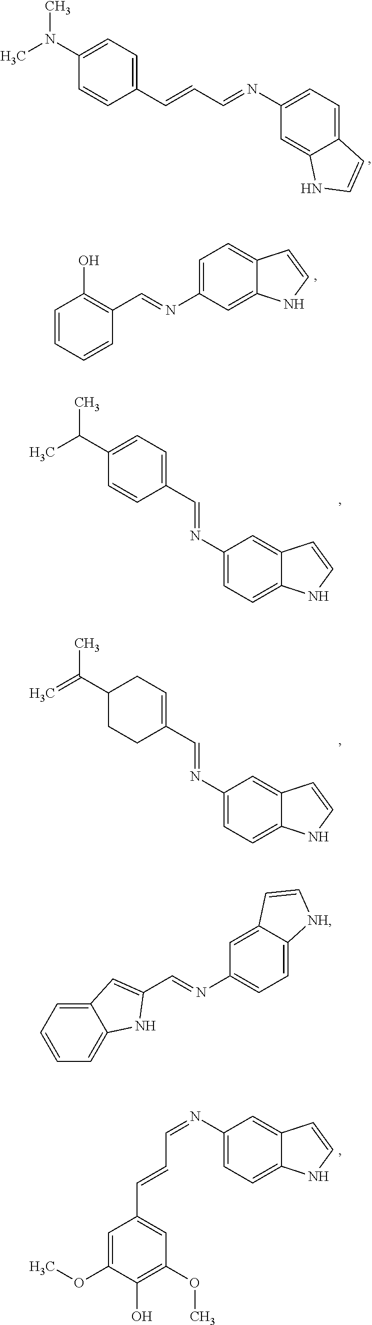

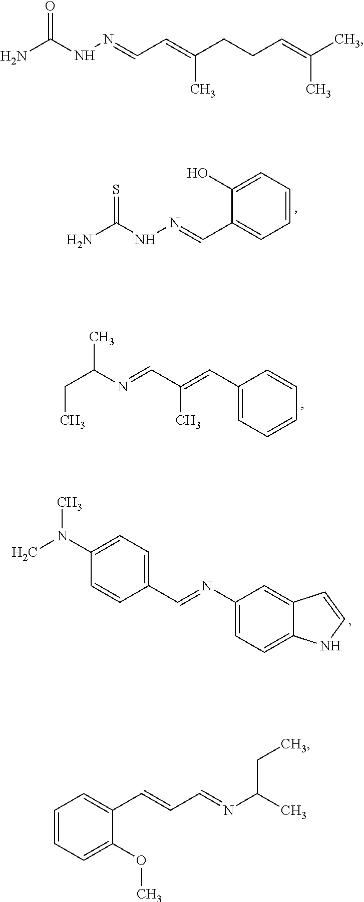 [SMILES: CC(C)=CCC/C(C)=C/C=N/NC(N)=O ; NC(=S)N/N=C/c1ccccc1O ; CCC(C)/N=C/C(C)=C/c1ccccc1 ; CN(C)c1ccc(/C=N/c2ccc3[nH]ccc3c2)cc1 ; CCC(C)/N=C/C=C/c1ccccc1OC]

27. A composition comprising a compound of formula: 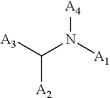
wherein A₁ is 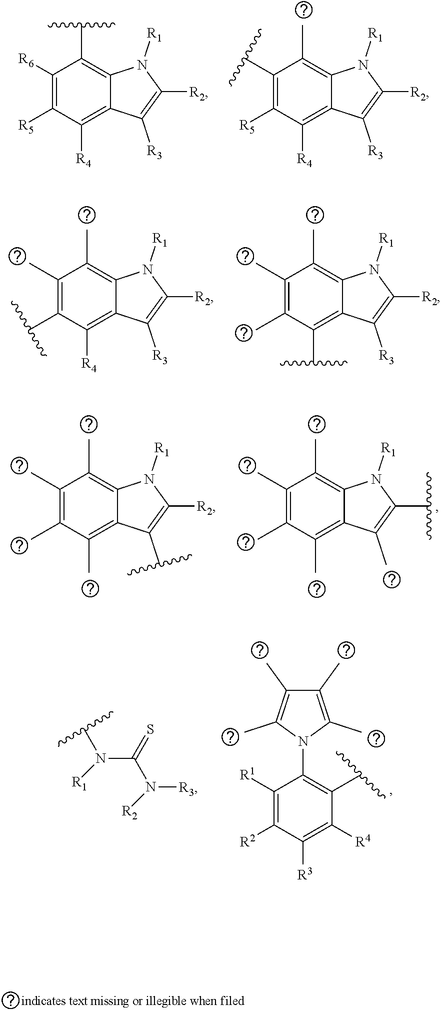
n-butyl, sec-butyl, iso-butyl, tert-butyl groups or a monocyclic heteroaryl group optionally substituted by one or more of R₁, R₂, R₃, R₄, R₅, R₆,
wherein, A2 and A3 are independently selected from
a) hydrogen;
b) 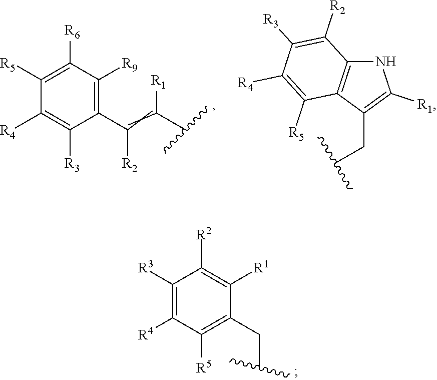
wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein A₄ is hydrogen or CHA₂A₃.

27. A composition comprising a compound of formula: 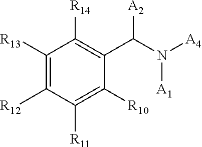
wherein A₁ is 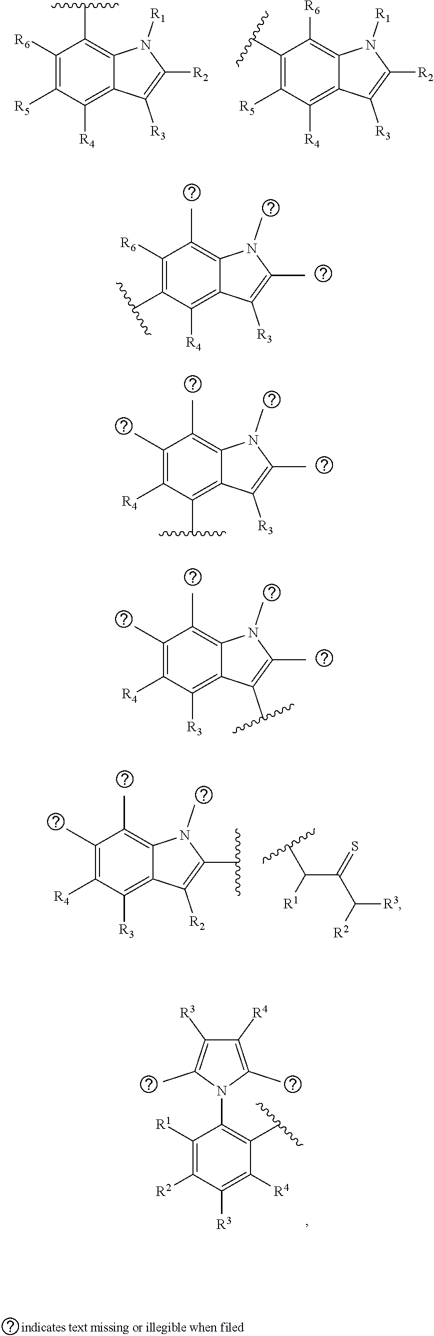
n-butyl, sec-butyl, iso-butyl, tert-butyl groups or a monocyclic heteroaryl group optionally substituted by one or more of R₁, R₂, R₃, R₄, R₅, R₆,
wherein, A₂ is independently selected from
a) hydrogen;
b) a saturated or unsaturated aliphatic group optionally substituted with one or more unsubstituted or substituted alkyl groups or aryl groups or an imine functional group bearing an A1, A2 or A3 substituent;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein at least one R₁₀, R₁₁, R₁₂, R₁₃, R₁₄ is a CHA₂(NA₁A₄) group, and the remaining are independently selected from H, alkyl, unsaturated alkyl, F, Cl, Br, I, NO₂, OH, amino, acyl, alkyl ether or aryl ether, monocyclic or polycyclic aryl or heteroaryl, group; and wherein R₇, R₈ are H or alkyl,
wherein A₄ is hydrogen or CHA₂A₃.

28. A method of treating a rubber polymer to protect it against damage by ozone comprising, treating the polymer with an effective amount of a composition comprising a compound of formula: 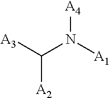
wherein A₁ is 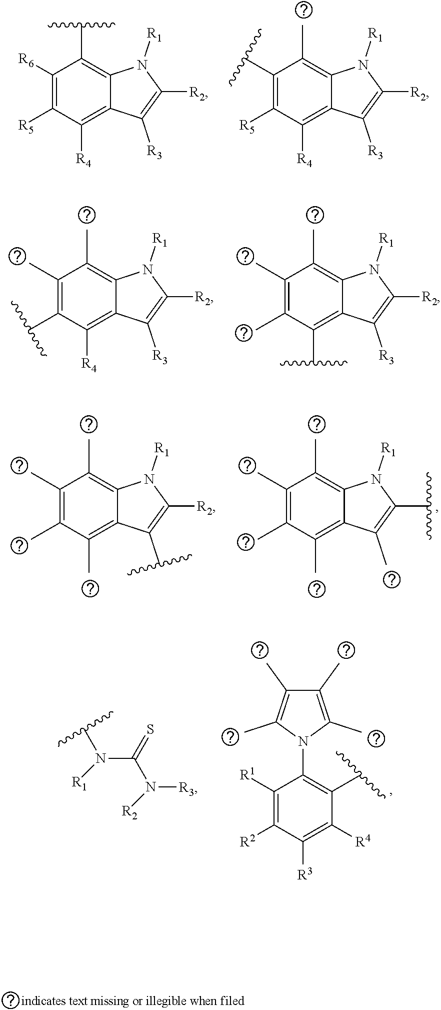
n-butyl, sec-butyl, iso-butyl, tert-butyl groups or a monocyclic heteroaryl group optionally substituted by one or more of R₁, R₂, R₃, R₄, R₅, R₆,
wherein, A2 and A3 are independently selected from
a) hydrogen;
b) 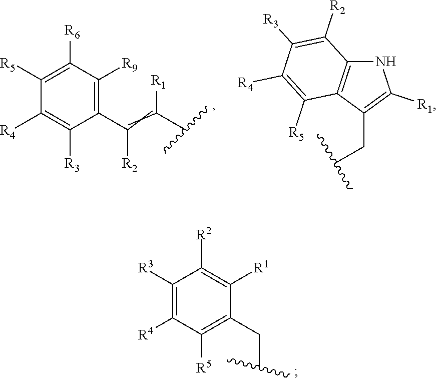
wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein A₄ is hydrogen or CHA₂A₃.

29. The method of claim 28 wherein the rubber is a natural rubber.

30. The method of claim 28 wherein the polymer is selected from at least one of polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber or mixtures thereof.

31. The method of claim 28 wherein the compound is present in an amount from 0.1 to 10 parts per 100 parts by weight of rubber.

32. The method of claim 28 wherein a mixture of 1 or more of the compounds is present in a total amount from 0.1 to 10 parts per 100 parts by weight of rubber.

33. The method of claim 28 wherein a mixture of 1 or more of the compounds present in a total amount of 0.1 to 5 parts per 100 parts by weight of rubber.

34. The method of claim 28 wherein a mixture of 1 or more of the compounds used in a vulcanized elastomeric article.

35. The method of claim 28 wherein a mixture of 1 or more of the compounds used in a vehicle tire.

36. A method of treating a rubber polymer to protect it against damage by ozone comprising, treating the polymer with an effective amount of a composition comprising a compound of formula: 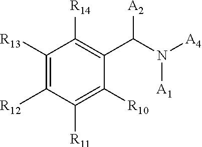
wherein A₁ is 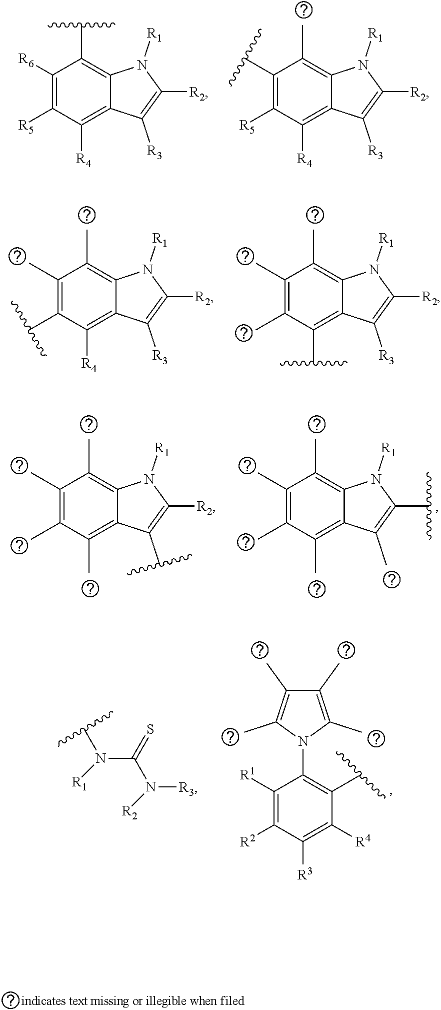
n-butyl, sec-butyl, iso-butyl, tert-butyl groups or a monocyclic heteroaryl group optionally substituted by one or more of R₁, R₂, R₃, R₄, R₅, R₆,
wherein, A₂ is independently selected from
a) hydrogen;
b) a saturated or unsaturated aliphatic group optionally substituted with one or more unsubstituted or substituted alkyl groups or aryl groups or an imine functional group bearing an A1, A2 or A3 substituent;
c) a monocyclic or polycyclic aryl or heteroaryl group optionally substituted with C1 to C20 straight or branched chain saturated alkyl, unsaturated alkyls, F, Cl, Br, I, NO₂, OH, NR₇R₈, acyl, alkyl ether or aryl ether substituents or an imine functional group bearing an A1, A2 or A3 substituent; wherein the aryl substituents are optionally substituted with one or more F, Cl, Br, I, NO2, OH, N R₇R₈, acyl, alkyl ether or aryl ether substituents;
wherein R₁, R₂, R₃, R₄, R₅, R₆, R₉ are independently selected from H, alkyl, unsaturated alkyl, F, Cl, Br, I, NO₂, OH, amino, acyl, alkyl ether or aryl ether, monocyclic or polycyclic aryl or heteroaryl group; and wherein R₇, R₈ are H or alkyl,
wherein at least one R₁₀, R₁₁, R₁₂, R₁₃, R₁₄ is a CHA₂(NA₁A₄) group, and the remaining are independently selected from H, alkyl, unsaturated alkyl, F, Cl, Br, I, NO₂, OH, amino, acyl, alkyl ether or aryl ether, monocyclic or polycyclic aryl or heteroaryl, group; and wherein R₇, R₈ are H or alkyl,
wherein A₄ is hydrogen or CHA₂A₃.

37. The method of claim 36 wherein the rubber is a natural rubber.

38. The method of claim 36 wherein the polymer is selected from at least one of polyisoprene rubber, polyisobutylene rubber, polybutadiene rubber, styrenebutadiene rubber, styrene-isoprene-butadiene rubber, styrene-isoprene rubber, butadiene-isoprene rubber, polybutadiene, butyl rubber, neoprene, ethylene-propylene rubber, ethylene-propylene-diene rubber (EPDM), acrylonitrile-butadiene rubber (NBR), silicone rubber, the fluoroelastomers, ethylene acrylic rubber, ethylene vinyl acetate copolymer (EVA), epichlorohydrin rubbers, chlorinated polyethylene rubbers, chlorosulfonated polyethylene rubbers, hydrogenated nitrile rubber, tetrafluoroethylene-propylene rubber or mixtures thereof.

39. The method of claim 36 wherein the compound is present in an amount from 0.1 to 10 parts per 100 parts by weight of rubber.

40. The method of claim 36 wherein a mixture of 1 or more of the compounds is present in a total amount from 0.1 to 10 parts per 100 parts by weight of rubber.

41. The method of claim 36 wherein a mixture of 1 or more of the compounds present in a total amount of 0.1 to 5 parts per 100 parts by weight of rubber.

42. The method of claim 36 wherein a mixture of 1 or more of the compounds used in a vulcanized elastomeric article.

43. The method of claim 28 wherein a mixture of 1 or more of the compounds used in a vehicle tire.
